(19) United States
(12) Patent Application Publication
(10) Pub. No.: US 2026/0088146 A1
(43) Pub. Date: Mar. 26, 2026

(54) MEDICAL VIRTUAL ASSISTANT

(71) Applicant: Align Technology, Inc., San Jose, CA (US)

(72) Inventors: Anamaria Castillo, Thornwood, NY (US); Pierre Velu, St. Cloud (FR); Vanessa Carpano Chauvin, EK Bergen (NL); Adrian Barry, Scottsdale, AZ (US); Eric Brown, Cary, NC (US); Christopher E. Cramer, Durham, NC (US); Niko Benjamin Huber, Zug (CH); Sravani Gurijala, Apex, NC (US); Ramesh Kothapalli, Hyderabad (IN); Nikita Singh, Hyderabad (IN); Udaya Kumar Swamy Vasa, Hyderabad (IN); Nikhil Kumar, Fuquay Varina, NC (US)

(21) Appl. No.: 19/337,760

(22) Filed: Sep. 23, 2025

Related U.S. Application Data
(60) Provisional application No. 63/698,401, filed on Sep. 24, 2024.
(60) Provisional application No. 63/725,981, filed on Nov. 27, 2024.

(51) Int. Cl. G16H 20/00; A61B 1/24; G10L 13/02; G10L 15/183; G10L 15/30

(52) U.S. Cl. CPC G16H 20/00; A61B 1/24; G10L 13/02; G10L 15/183; G10L 15/30

(57) ABSTRACT

A method includes receiving, by a user device, a prompt associated with treatment of a patient. The method includes determining a treatment context for the treatment of the patient based on access to a back-end treatment context support system. The method includes processing the prompt in view of the treatment context using one or more trained machine learning models to generate one or more actionable recommendations. The method includes outputting the one or more actionable recommendations via at least one of the user device or an additional device associated with the user device.

### RELATED APPLICATIONS

This patent application claims the benefit under 35 U.S.C. § 119(e) of U.S. Provisional Application No. 63/698,401, filed Sep. 24, 2024, and further claims the benefit under 35 U.S.C. § 119(e) of U.S. Provisional Application No. 63/725,981, filed Nov. 27, 2024, both of which are incorporated by reference herein.

### TECHNICAL FIELD

Embodiments of the present disclosure relate to the fields of medicine and artificial intelligence and, in particular, to a medical virtual assistant. Embodiments of the present disclosure also relate to the field of medicine, and in particular medical applications. Embodiments of the present disclosure further relate to an application-agnostic messaging platform usable with medical applications and other applications.

### BACKGROUND

Medical software such as treatment planning software, intraoral scanning software, treatment management software, and so on can be complex and difficult to use. Moreover, it is important that such software be used correctly in order to ensure that patients receive the best possible care. Currently, when a doctor or other medical practitioner has questions about how to use medical software or about a patient treatment associated with medical software, the doctor or other medical practitioner either calls a hotline for the medical software, interfaces with a chatbot online, or reviews documentation about the medical software. Each of these options requires the doctor or other medical practitioner to stop other activities and to call the medical software provider, access a chatbot provided by the medical software provider via a computer, or manually perform research. Accordingly, getting answers about medical treatment associated with medical software can be inconvenient and time consuming. Additionally, a doctor cannot obtain such answers while chairside with a patient.

Furthermore, traditionally, a doctor and patient communicate with one another via face-to-face consultations, in which the patient visits the doctor at a doctor office or the doctor makes a house call to visit a patient. Patients and doctors may also sometimes communicate over the phone, generally to discuss test results, clarify medical instructions, or address urgent health concerns that don't require an in-person visit. Virtual care, also known as telemedicine or telehealth, also provides a mechanism for doctors and patients to meet virtually over specially designed web-based digital platforms, in which the doctor and patient each log into the web-based digital platform for a real-time face-to-face virtual consultation by taking advantage of video cameras, microphones and speakers of the doctor's computer and the patient's computer. Some healthcare organizations also offer mobile apps that the doctor and/or patient can install on their mobile devices. Like the web-based digital platforms, the mobile apps may enable a doctor and a patient, each having a copy of the mobile app installed on their mobile device, to set up and hold a virtual appointment.

### SUMMARY

In a 1^{st} aspect of the disclosure, a method comprises: receiving, by a user device, a prompt associated with treatment of a patient; determining a treatment context for the treatment of the patient based on access to a back-end treatment context support system; processing the prompt in view of the treatment context using one or more trained machine learning models to generate one or more actionable recommendations; and outputting the one or more actionable recommendations via at least one of the user device or an additional device associated with the user device.

In a 2^{nd} aspect of the disclosure, a system comprises: a local computing device configured to execute a medical application for a patient; a user device comprising a microphone and a speaker, the user device configured to: capture a prompt asking associated with treatment of the patient; and a server computing device configured to: receive the prompt from the user device; receive one or more clues about a treatment context from at least one of the user device or the local computing device; determine the treatment context for the patient based on the one or more clues; process the prompt in view of the treatment context using one or more trained machine learning models to generate one or more actionable recommendations; and send the one or more actionable recommendations to at least one of the user device or the local computing device, wherein the one or more actionable recommendations are output via at least one of the user device or the local computing device.

In a 3^{rd} aspect of the disclosure, a system comprises: a server computing device comprising a messaging platform, wherein the messaging platform is configured to provide messaging between a plurality of different types of medical applications; a first computing device of a doctor, the first computing device comprising a doctor-focused medical application and a first chat module that integrates with the doctor-focused medical application and that enables the doctor-focused medical application to interface with the messaging platform; and a second computing device of a patient or prospective patient, the second computing device comprising a patient-focused medical application and a second chat module that integrates with the patient-focused medical application and that enables the patient-focused medical application to interface with the messaging platform, wherein the patient-focused medical application has different functionality than the doctor-focused medical application; wherein the messaging platform is configured to send messages between the doctor-focused medical application of the first computing device and the patient-focused medical application of the second computing device.

In a 4^{th} aspect of the disclosure, a mobile computing device of a patient comprises: a storage device comprising instructions for a medical application; a microphone; and one or more processing devices configured to: receive a voice instruction associated with a function of the medical application via the microphone; determine that the voice instruction is for the function of the medical application; cause the medical application to perform the function; and generate an output responsive to causing the medical application to perform the function.

### BRIEF DESCRIPTION OF THE DRAWINGS

Embodiments of the present disclosure are illustrated by way of example, and not by way of limitation, in the figures of the accompanying drawings.

FIG. 1 illustrates a system for providing a medical virtual assistant to a medical practice, in accordance with an embodiment.

FIG. 2 is a flow diagram for a method of providing a medical virtual assistant, in accordance with an embodiment.

FIG. 3 is a flow diagram for a further method of providing a medical virtual assistant, in accordance with an embodiment.

FIG. 4 is a sequence diagram for a method of engaging with a user using a medical virtual assistant, in accordance with an embodiment.

FIG. 5 is a flow diagram for a method of engaging with a user using a medical virtual assistant, in accordance with an embodiment.

FIG. 6 illustrates a large language model that, in combination with additional logic, may function as a medical virtual assistant, in accordance with an embodiment.

FIG. 7A illustrates a system that enables communication between doctors and patients, in accordance with an embodiment.

FIG. 7B illustrates an application architecture for a messaging platform, in accordance with an embodiment.

FIG. 8A is a sequence diagram illustrating bidirectional communication between disparate medical applications via an application-agnostic messaging platform, in accordance with an embodiment.

FIG. 8B is an additional sequence diagram illustrating bidirectional communication between disparate medical applications via an application-agnostic messaging platform, in accordance with an embodiment.

FIG. 9 is a sequence diagram illustrating secured bidirectional communication between disparate medical applications via an application-agnostic messaging platform, in accordance with an embodiment.

FIG. 10 is a flow diagram for a method of providing a live chat session between disparate medical applications, in accordance with an embodiment.

FIG. 11 illustrates a mobile device with a voice-controlled medical application installed thereon, in accordance with an embodiment.

FIG. 12 is a flow diagram for a further method of providing voice control of a medical application, in accordance with an embodiment.

FIG. 13 illustrates a block diagram of an example computing device, in accordance with embodiments of the present disclosure.

### DETAILED DESCRIPTION

Described herein are embodiments of a medical virtual assistant. The medical virtual assistant is a virtual assistant that receives prompts (e.g., voice and/or text prompts) associated with one or more medical operations, medical products and/or medical software, determines appropriate responses to the prompts, and outputs the determined responses. The responses may include text replies to prompts, speech replies to prompts, and/or performance of actions such as updating medical records, updating a treatment plan, scheduling an appointment, ordering a medical product (e.g., such as a set of orthodontic aligners), controlling a medical application, explaining how to use a medical application, and so on. In embodiments, the medical virtual assistant is tuned to one or more medical practices (e.g., orthodontic and/or dental practices) and/or one or more medical product and/or service providers. The medical virtual assistant may include or be provided by a combination of a clinically accurate large language model (LLM) and a back-end treatment context support system. The back-end treatment context support system may include one or more clinical context data stores that store clinical context information such as historical patient information, current patient information, medical product information, medical application information, and so on, a search engine that can perform searches in the data store(s) for clinical context information, and/or an LLM optimized to generate search queries to be input into the search engine. Clinical context may include information on a particular patient (e.g., a patient that a doctor is working on). Clinical context may additionally and/or alternatively include context of procedures/operations that a doctor is performing on a patient. In embodiments, the search engine may perform searches of the data store(s) based on one or more treatment context clues (e.g., which may be gathered by a chat model and/or the LLM). The search may enable the search engine to identify, for example, a current patient that the doctor is working on/seeing without the doctor explicitly informing the medical virtual assistant of an identity of the patient. Then, any questions addressed to the medical virtual assistant may be answered with respect to the determined patient at hand. The medical virtual assistant may reduce the friction between a medical product and/or medical services provider and customers (e.g., doctors, dentists, patients, orthodontists, etc.) of the medical product and/or medical services provider. The medical virtual assistant may make it faster and easier to access a wealth of clinical data that is available in the clinical context data store(s) to help doctors and their staff be more clinically confident using and/or providing one or more medical products and/or medical services. The medical virtual assistant may additionally deliver medical practice efficiency gains by connecting customers (e.g., high volume customers) with increased clinical support, customer support and/or field support faster and easier than is presently achievable.

In embodiments, the medical virtual assistant is paired with one or more user devices, such as a wearable internet of things (IoT) device (e.g., a pin) that is wearable by a user, an augmented reality display (e.g., AR glasses), a standalone IoT table top device (e.g., such as a smart speaker), an application (app) for a mobile phone, tablet computer, laptop computer, and/or smart television (TV), an IoT device embedded in a medical device (e.g., such as an intraoral scanner), and so on. Via the one or more user devices, users may generate voice and/or text prompts. The voice and/or text prompts may then be sent to the medical virtual assistant, which may determine a treatment context associated with the received prompt (e.g., based on determining one or more context clues and accessing treatment context or clinical context information from one or more clinical context data stores using the determined context clues), and generate clinically accurate answers to the prompts in view of the determined treatment context.

Embodiments provide for a system that incorporates several components including a physical input/output device (e.g., a user device) that can receive prompts and output generated responses to the prompts, an artificial intelligence (e.g., LLM) based intent system, and an “intelligent” content management/delivery system capable of identifying treatment context that can be used to ensure that answers to received prompts are clinically accurate. Responsive to user requests for information received via the user device, answers to questions may be provided also through the user device and/or via other media (e.g., such as an email, text, phone call, etc. to another user device that may be different from the user device from which the user requests were received).

In embodiments, the medical virtual assistant saves doctors and their staff time and hassle by providing quick and easy access to the information and support they might need. In embodiments, the medical virtual assistant enhances customer experience with medical products and services by providing personalized and tailored answers and solutions immediately. In embodiments, the medical virtual assistant empowers customers of medical products/services to make informed decisions and take action by providing clear and concise guidance and instructions. In embodiments, the medical virtual assistant leap-frogs typical “omnichannel” communications to a truly digital channel that is interactive, optionally with status lights denoting waiting messages. Accordingly, in embodiments alerts to doctors and their staff may never be missed. In embodiments, the medical virtual assistant reduces customer support and/or clinical support friction to near zero. For example, the medical virtual assistant may eliminate a need to look for a phone number to call to get advice from a medical product and/or service provider, and may eliminate a need for a doctor or their staff to find a computer and engage in text chats via the computer.

Also described herein are embodiments of an application-agnostic messaging platform that enables both live and asynchronous communication between disparate medical applications, such as between a doctor-focused medical application used by a doctor and a patient-focused medical application used by a patient. Any medical application and/or other application may connect with the messaging platform and exchange messages with other devices also connected to the messaging platform via a chat module that can be added to the medical application and/or other application. A version of the chat module may be added to many disparate medical applications to enable users of those disparate medical applications to communicate over the messaging platform. The messaging platform may provide secure communications, and may encrypt all messages between parties (e.g., between a doctor and a patient) to ensure that the communications remain private. The messaging platform may additionally enable transmission of files, images, videos, documents, and so on. For example, the messaging platform may enable patients to send images of themselves to the doctor for assessment. In some embodiments, the doctor interfaces with the messaging platform and/or medical application using a wearable IoT device (e.g., wearable pin) or AR display. In some embodiments, the doctor provides speech input, which is converted to text via a speech to text system (e.g., which may include one or more artificial intelligence (AI) models). The text may then be input to the messaging platform (e.g., and sent to a computing device of a patient).

In contrast to virtual care applications, the messaging platform enables users of multiple different medical applications to communicate with one another. Additionally, other types of applications may also connect to the messaging platform to enable users of those other types of applications to communicate with users of the medical applications. For example, sales representatives or business representatives of a medical device company may use an application with an integrated chat module to communicate with a doctor that uses a doctor-focused medical application and/or with a patient that uses a patient-focused medical application. Accordingly, the messaging platform provides increased flexibility over the monolithic virtual care systems provided by healthcare providers.

In embodiments, the messaging platform saves doctors and their staff time and hassle by providing quick and easy access to their patients, and to chat histories with their patients. In embodiments, the messaging platform enhances customer experience with medical products and services by providing live messaging between doctors and sales or service representatives of medical device companies. In embodiments, the messaging platform enables patients and doctors to easily and quickly communicate. The messaging platform leap-frogs typical “omnichannel” communications to a truly digital channel that is interactive, optionally with status lights denoting waiting messages. Accordingly, in embodiments alerts to doctors and their staff, as well as alerts to patients, may never be missed. In embodiments, the messaging platform reduces customer support and/or clinical support friction to near zero. For example, the messaging platform may eliminate a need to look for a phone number to call to get advice from a medical product and/or service provider.

Also described herein are embodiments of a voice-controlled medical application. In embodiments, the voice-controlled medical application is a patient-focused medical application, such as a virtual care medical application. Alternatively, the medical application may be a doctor-focused medical application. In one embodiment, the voice-controlled medical application is an orthodontic virtual care application that patients can use to track their orthodontic treatment, determine when to advance to a next treatment stage (e.g., when to use a new orthodontic aligner), when to visit their doctor for an in-person checkup, and so on. In embodiments, voice controls are added to a mobile device on which the medical application is installed. Even when the medical application is not running, a user may provide a voice command to the mobile device using a microphone of the user device to launch the medical application and execute a particular functionality of the medical application, or execute the particular functionality of the medical application without first launching the medical application (e.g., if the medical application is already running or a widget for the particular functionality of the medical application is installed on the mobile device). Addition of voice controls for the medical application, in particular for an orthodontic virtual care medical application, can make it much easier for a user to use functions of the medical application that will improve patient experience and/or treatment results. For example, the voice controls may be used to start and stop a timer that times how long an orthodontic aligner has been removed from the patient's mouth, to determine when a current aligner should be replaced with a next aligner, and so on.

Various embodiments are described herein. It should be understood that these various embodiments may be implemented as stand-alone solutions and/or may be combined. Accordingly, references to an embodiment, or one embodiment, may refer to the same embodiment and/or to different embodiments. Some embodiments are discussed herein with reference to intraoral scans and intraoral images. However, it should be understood that embodiments described with reference to intraoral scans also apply to lab scans or model/impression scans. A lab scan or model/impression scan may include one or more images of a dental site or of a model or impression of a dental site, which may or may not include height maps, and which may or may not include color images.

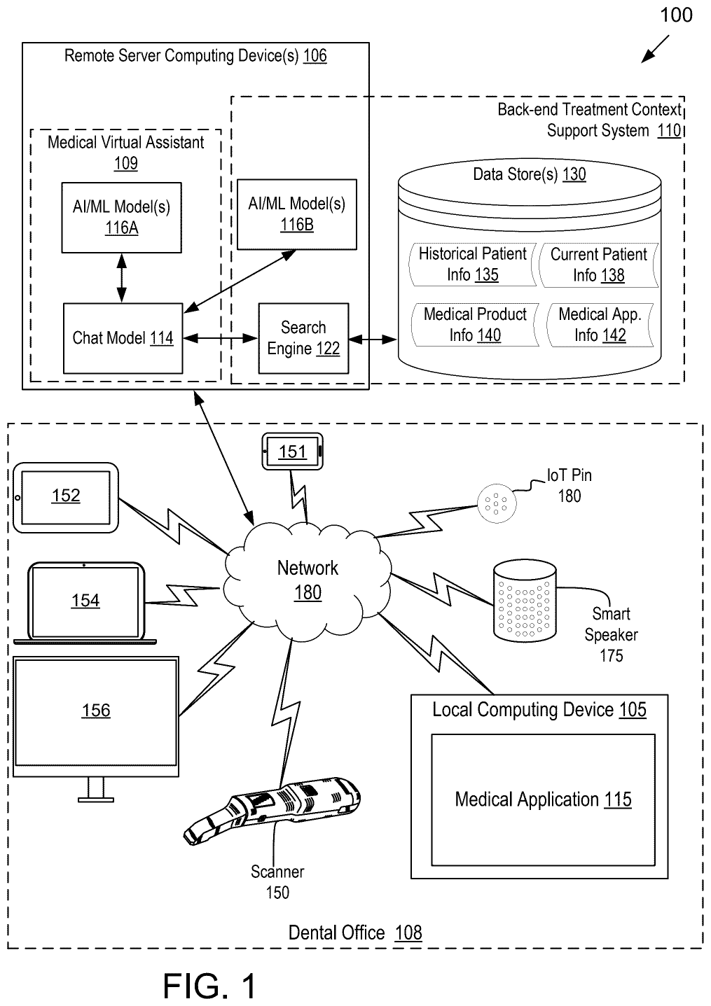

FIG. 1 illustrates a system 100 for providing a medical virtual assistant 109 to a medical practice, in accordance with an embodiment. In embodiments, the system 100 is configured to provide a medical virtual assistant 109 to one or more users (e.g., a doctor and their staff) in a dental office 108.

The system may include one or more components at a dental office 108 and one or more components or a medical virtual assistant 109 and of a back-end treatment context support system 110 that may be located remote from the dental office 108. The dental office 108 may include one or more user devices. The user devices may be, for example, a wearable internet of things (IoT) device (e.g., an IoT pin 180) that is wearable by a user, an augmented reality (AR) display (e.g., AR glasses), a standalone IoT table top device (e.g., such as a smart speaker 175), a mobile phone 151, a tablet computer 152, a laptop computer 154, a smart TV 156, an intraoral scanner 150, a local computing device 105 (e.g., such as a computing device of an intraoral scanning system that pairs with intraoral scanner 150), and so on. In embodiments, the mobile phone 151, tablet computer 152, laptop computer 154, smart TV 156, etc. may execute an application (app) configured to provide an interface to the medical virtual assistant 109. The application may leverage a microphone and/or speaker of the mobile phone 151, tablet computer 152, laptop computer 154, smart TV 156, etc. to receive verbal prompts from users and/or to provide verbal outputs responsive to the verbal inputs in some embodiments. In embodiments, smart speaker 175 is a physical desktop device comprising a power supply (e.g., a wired power supply for plugging into a wall outlet or a battery power supply including a rechargeable battery), a speaker, a microphone, and a network adapter (e.g., a wired or wireless network adapter for connecting to network 180).

In embodiments, medical virtual assistant 109 and back-end treatment context support system 110 may include one or more remote server computing devices 106 and one or more data stores 130 (e.g., which may be clinical context and/or treatment context data stores). The remote server computing device(s) 106 may host software that in combination provides a medical virtual assistant 109 that is accessible from the user devices in the dental office 108. In embodiments, the remote server computing device(s) 106 host a chat model 114, one or more machine learning (ML) models (also referred to as LLMs) 116A-B and/or other artificial intelligence (AI) models, and/or a search engine 122 configured to perform searches on one or more data stores 130 for treatment context and/or clinical context information. In embodiments, medical virtual assistant 109 is a virtual clinical assistant that uses one or more LLMs and a medical product/service provider's data set of clinical data, publicly available education data, product usage data and/or other clinical data related to one or more medical fields (e.g., fields of orthodontics and/or restorative dentistry) to provide assistance to doctors and their staff.

In embodiments, remote server computing device(s) 106 include devices associated with a cloud computing service. Cloud computing services provide on-demand access to computing resources over the internet, including servers, storage, databases, networking, software, analytics, and intelligence. Cloud computing uses virtualization technology to divide physical hardware into multiple virtual machines (VMs). Each VM can run its own operating system and applications independently, allowing multiple users or organizations to share the same physical resources. The cloud computing service may include an infrastructure as a service (IaaS) that provides virtualized computing resources, a platform as a service (PaaS) that offers a platform on which applications can be developed, run and managed, and/or software as a service (SaaS) that delivers software applications over a network.

The remote server computing device(s) 106 and/or data store(s) 130 may be connected to one or more user devices in the dental office 108 via a network 180. The network 180 may be a local area network (LAN), a public wide area network (WAN) (e.g., the Internet), a private WAN (e.g., an intranet), a wireless network of a wireless carries (e.g., a 3G, 4G or 5G wireless network), or a combination thereof.

Computing device 105 may be coupled to and/or include a data store (not shown), which may be local data store and/or a remote data store. Computing device 105 and remote server computing device(s) 106 may each include one or more processing devices, memory, secondary storage, one or more input devices (e.g., such as a keyboard, mouse, tablet, and so on), one or more output devices (e.g., a display, a printer, etc.), and/or other hardware components.

In some embodiments, dental office 108 includes an intraoral scanning system that includes intraoral scanner 150 and local computing device 105. In embodiments, a handheld intraoral scanner 150 (also referred to as an intraoral scanner or simply a scanner) is connected to local computing device 105 either wirelessly or via a wired connection. In one embodiment, scanner 150 is wirelessly connected to computing device 105 via a direct wireless connection. In one embodiment, scanner 150 is wirelessly connected to computing device 105 via a wireless network. In one embodiment, the wireless network is a Wi-Fi network. In one embodiment, the wireless network is a Bluetooth network, a Zigbee network, or some other wireless network. In one embodiment, the wireless network is a wireless mesh network, examples of which include a Wi-Fi mesh network, a Zigbee mesh network, and so on. In an example, local computing device 105 may be physically connected to one or more wireless access points and/or wireless routers (e.g., Wi-Fi access points/routers). Intraoral scanner 150 may include a wireless module such as a Wi-Fi module, and via the wireless module may join the wireless network via the wireless access point/router.

Dental office 108 may further include one or more displays 156 (also referred to as smart TVs), which optionally may be operatively connected to computing device 105. Some displays 156 may be physically connected to the computing device 105 via a wired connection. Some displays 156 may be wirelessly connected to computing device 105 via a wireless connection, which may be a direct wireless connection or a wireless connection via a wireless network. In embodiments, display 156 is a smart display such as a smart television (TV). A smart TV may include an application installed thereon for interfacing with a medical virtual assistant provided by back-end treatment context support system 110. Alternatively, or additionally, a smart TV may include a web browser, which may be used to navigate to a web page that accesses the medical virtual assistant 109.

Dental office 108 may further include one or more additional computing devices such as tablet computer 152, laptop computer 154, mobile phone 151, etc. In embodiments, one or more computing devices may be mobile computing devices such as laptops, notebook computers, tablet computers, mobile phones, portable game consoles, and so on. In embodiments, one or more computing devices may be traditionally stationary computing devices, such as desktop computers, set top boxes, game consoles, and so on.

Some user devices (e.g., display 156, mobile phone 151, tablet computer 152, laptop computer 154, computing device 105, etc.) may include applications that interface with remote server computing devices 106 of the back-end treatment context support system 110 to provide access to a medical virtual assistant 109. Alternatively, one or more of these user devices may access the medical virtual assistant 109 using a web browser.

Intraoral scanner 150 may be a wireless handheld device that is not tethered to a computer, display, and/or other hardware. Alternatively, intraoral scanner 150 may have a wired connection to local computing device. The intraoral scanner 150 may be used to perform intraoral scanning of a patient's oral cavity.

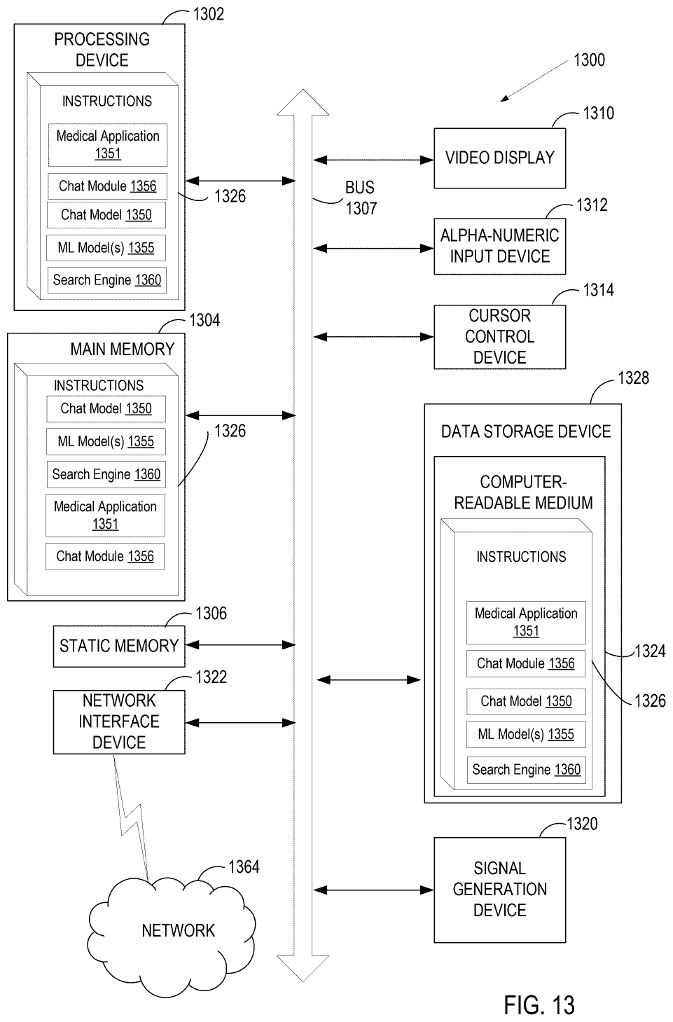

Intraoral scanner 150 may include one or more light source, optics and one or more detectors for generating intraoral scan data (e.g., intraoral scans, color images, NIRI images, etc.), one or more buttons and/or touch sensitive inputs (e.g., touch pads and/or touchscreens), an inertial measurement unit (IMU), and so on. Intraoral scanner 150 may additionally include a memory and/or a processing device (e.g., a controller) for performing initial processing on some or all of the intraoral scan data before it is transmitted to local server computing device 105. Scanner 150 may additionally include a communication module (e.g., a wireless communication module and/or a wired communication module) such as a network interface controller (NIC) capable of communicating via Wi-Fi, via an Ethernet connection, via an InfiniBand connection, via a universal serial bus (USB) connection, via third generation (3G), fourth generation (4G) and/or fifth generation (5G) telecommunications protocols (e.g., global system for mobile communications (GSM), long term evolution (LTE), Wi-Max, code division multiple access (CDMA), etc.), via Bluetooth, via Zigbee, and/or via other wireless protocols. Alternatively, or additionally, the scanner 150 may connect to a wide area network (WAN) such as the Internet, and may connect to the local computing device 105 and/or remote server computing device 106 via the WAN. One example of a scanner 150 is the iTero® intraoral digital scanner manufactured by Align Technology, Inc. Another example of a scanner 150 is set forth in U.S. Publication No. 2019/0388193, filed Jun. 19, 2019, which is incorporated by reference herein. Two example scanners are described in greater detail below with reference to FIGS. 13-14.

Intraoral scanner 150 may generate intraoral scans, which may be or include color or monochrome 3D information, and send the intraoral scans to local computing device 105 via the wireless or wired connection. Intraoral scanner 150 may additionally or alternatively generate color two-dimensional (2D) images (e.g., viewfinder images), and send the color 2D images to local server computing device 105 via the wireless connection. Scanner 150 may additionally or alternatively generate 2D or 3D images under certain lighting conditions, such as under conditions of infrared or near-infrared (NIRI) light and/or ultraviolet light, and may send such 2D or 3D images to server computing device 105 via the wireless connection. Intraoral scans, color images, and images under specified lighting conditions (e.g., NIRI images, infrared images, ultraviolet images, etc.) are collectively referred to as intraoral scan data. An operator may start recording scans with the scanner 150 at a first position in the oral cavity, move the scanner 150 within the oral cavity to a second position while the scans are being taken, and then stop recording the scans.

Local computing device 105 may include a medical application 115. In some embodiments, medical application 115 is an intraoral scan application 115. Alternatively, medical application 115 may be a treatment planning application, a treatment management application, and/or other type of medical application.

In embodiments where local computing device 105 and scanner 150 are components of an intraoral scanning system, the local computing device 105 and scanner may operate together to effectuate an intraoral scan. A result of the intraoral scan may be intraoral scan data that may include one or more sets of intraoral scans, one or more sets of viewfinder images (e.g., color 2D images showing a field of view of the intraoral scanner), one or more sets of NIRI images, and so on. Each intraoral scan may be a two-dimensional (2D) or 3D image that includes a height information (e.g., a height map) of a portion of a dental site, and thus may include x, y and z information. In one embodiment, each intraoral scan is a point cloud.

In embodiments, scanner 150 generates and sends to computing device 105 a stream of intraoral scan data. The stream of intraoral scan data may include separate streams of intraoral scans, color images and/or NIRI images (and/or other images under specific lighting conditions) in some embodiments. Local computing device 105 receives intraoral scan data from scanner 150, then stores the intraoral scan data in a data store.

According to an example, a user (e.g., a practitioner) may subject a patient to intraoral scanning. In doing so, the user may apply scanner 150 to one or more patient intraoral locations. The scanning may be divided into one or more segments. As an example, the segments may include a lower dental arch of the patient, an upper dental arch of the patient, one or more preparation teeth of the patient (e.g., teeth of the patient to which a dental device such as a crown or other dental prosthetic will be applied), one or more teeth which are contacts of preparation teeth (e.g., teeth not themselves subject to a dental device but which are located next to one or more such teeth or which interface with one or more such teeth upon mouth closure), and/or patient bite (e.g., scanning performed with closure of the patient's mouth with the scan being directed towards an interface area of the patient's upper and lower teeth). Via such scanner application, the scanner 150 may provide intraoral scan data to computing device 105. The intraoral scan data may be provided in the form of intraoral scan/image data sets, each of which may include 2D intraoral scans/images and/or 3D intraoral scans/images of particular teeth and/or regions of an intraoral site. In one embodiment, separate scan/image data sets are created for the maxillary arch, for the mandibular arch, for a patient bite, and for each preparation tooth. Alternatively, a single large intraoral scan/image data set is generated (e.g., for a mandibular and/or maxillary arch). Such scans/images may be provided from the scanner to the computing device 105 in the form of one or more points (e.g., one or more pixels and/or groups of pixels). For instance, the scanner 150 may provide such a 3D scan/image as one or more point clouds.

During an intraoral scan session, medical application 115 may receive and processes intraoral scan data (e.g., intraoral scans) and generate a 3D surface of a scanned region of an oral cavity (e.g., of a dental site) based on such processing. To generate the 3D surface, medical application 115 may register and “stitch” or merge together the intraoral scans generated from the intraoral scan session in real time or near-real time as the scanning is performed. In one embodiment, performing registration includes capturing 3D data of various points of a surface in multiple scans (views from a camera), and registering the scans by computing transformations between the scans. The 3D data may be projected into a 3D space for the transformations and stitching. The scans may be integrated into a common reference frame by applying appropriate transformations to points of each registered scan and projecting each scan into the 3D space.

In one embodiment, registration is performed for adjacent or overlapping intraoral scans (e.g., each successive frame of an intraoral video). In one embodiment, registration is performed using blended scans and/or reduced or cropped scans. Registration algorithms are carried out to register two or more adjacent intraoral scans and/or to register an intraoral scan with an already generated 3D surface, which essentially involves determination of the transformations which align one scan with the other scan and/or with the 3D surface. Registration may involve identifying multiple points in each scan (e.g., point clouds) of an scan pair (or of a scan and the 3D model), surface fitting to the points, and using local searches around points to match points of the two scan (or of the scan and the 3D surface). For example, medical application 115 may match points of one scan with the closest points interpolated on the surface of another image, and iteratively minimize the distance between matched points. Other registration techniques may also be used. Intraoral scan application 115 may repeat registration and stitching for all scans of a sequence of intraoral scans and update the 3D surface as the scans are received.

When a scan session is complete (e.g., all scans for an intraoral site or dental site have been captured), a model generator of medical application 115 may generate a virtual 3D model (also referred to as a digital 3D model) of one or more scanned dental sites. The virtual 3D model includes a 3D surface of the one more scanned dental sites, but has a higher degree of accuracy than the 3D surface generated during the scanning process. To generate the virtual 3D model, medical application 115 may register and “stitch” or merge together the intraoral scans generated from the intraoral scan session. In one embodiment, registration is performed for adjacent and/or overlapping intraoral scans (e.g., each successive frame of an intraoral video). In one embodiment, registration is performed using blended scans and/or reduced or cropped scans. Registration algorithms may be carried out to register two or more adjacent intraoral scans and/or to register an intraoral scan with a 3D model, which essentially involves determination of the transformations which align one scan with the other scan and/or with the 3D model. Registration may involve identifying multiple points in each scan (e.g., point clouds) of a scan pair (or of a scan and the 3D model), surface fitting to the points, and using local searches around points to match points of the two scans (or of the scan and the 3D model). For example, medical application 115 may match points of one scan with the closest points interpolated on the surface of another scan, and iteratively minimize the distance between matched points. Other registration techniques may also be used. The registration and stitching that are performed to generate the 3D model may be more accurate than the registration and stitching that are performed to generate the 3D surface that is shown in real time or near-real time during the scanning process.

Medical application 115 may repeat registration for all scans of a sequence of intraoral scans to obtain transformations for each scan, to register each scan with the previous one and/or with a common reference frame (e.g., with the 3D model). Medical application 115 integrates all scans into a single virtual 3D model by applying the appropriate determined transformations to each of the scans. Each transformation may include rotations about one to three axes and translations within one to three planes.

Once a 3D model of a dental site (e.g., a dental arch) is generated, medical application 115 may generate a view of the 3D model and output the view to its own display and/or to a display of an appropriate device 152-156 for display of the 3D model to a user (e.g., a doctor). A doctor may then interface with the device to generate commands to change the view of the 3D model (e.g., by zooming in or out, panning, rotating, etc.). A user interface of the medical application 115 enables users to interact with medical application 115 through manipulation of graphical elements such as graphical icons and visual indicators such as buttons, menus, and so on, through text input, and so on. Medical application 115 may include a number of modes, such as a planning mode, a scan mode, an image processing mode, and a delivery mode. The user interface may display different graphical elements for each of the various modes.

In one embodiment, medical application 115 includes a treatment planner configured to perform treatment planning for orthodontic treatment and/or prosthodontic treatment. The treatment planner may additionally perform dental diagnostics and/or prognostics. Via the user interface of the treatment planner, a practitioner may view one or more of the upper dental arch, the lower dental arch, a particular preparation tooth and/or the patient bite, each of which may be considered a separate scan segment or mode. The treatment planner in embodiments generates an orthodontic treatment plan, including a 3D model for a final tooth arrangement and 3D models for one or more intermediate tooth arrangements. The treatment planner may additionally or alternatively perform diagnostics of a patient's oral cavity and/or provide a prognosis of one or more dental conditions and/or suggested treatments for the one or more dental conditions. The treatment planner may further perform one or multiple different analyses of the patient's dental arches and/or bite. The analyses may include an analysis for identifying tooth cracks, an analysis for identifying gum recession, an analysis for identifying tooth wear, an analysis of the patient's occlusal contacts, an analysis for identifying crowding of teeth (and/or spacing of teeth) and/or other malocclusions, an analysis for identifying plaque, an analysis for identifying tooth stains, an analysis for identifying caries, and/or other analyses of the patient's dentition. Once the analyses are complete, a dental diagnostics summary and/or detailed dental diagnostics information optionally including prognosis and/or treatment options may be presented. A doctor may control the treatment planner and navigate menus and options of the treatment planner's user interface.

In a non-limiting example, a patient who wishes to straighten their teeth may opt for Invisalign® treatment. Invisalign is a process that creates a custom made series of clear aligners specifically for the patient. The clear aligners are worn over the patient's teeth and gradually shift the patient's teeth. A new set of aligners may be worn after a specified period of time (e.g., two weeks) until treatment is complete.

The patient may visit a dental practitioner or orthodontist to begin Invisalign treatment. The dental practitioner may utilize a scanning system including scanner 150 and local computing device 105 to scan the patient's teeth in a scanning mode. The dental practitioner may use scanner 150 to capture the patient's teeth segments (e.g., upper arch, lower arch, bite segments) in one or more sets of intraoral scans. The medical application 115 may register and stitch together the intraoral scans to create a 3D rendering of the scanned segments and present the 3D rendering to the dental practitioner on the user interface of the medical application. Once the scans are completed, the dental practitioner may next navigate to an image processing mode, which may generate a virtual 3D model by registering and stitching together the intraoral images. Once an adequate set of 3D renderings and/or virtual 3D model are complete, the 3D renderings and/or 3D models may be saved to the patient profile.

The dental practitioner may then provide input to switch to a planning mode, in which a final tooth arrangement may be determined and one or more intermediate tooth arrangements may be determined. A treatment plan may be generated to provide a progression of treatment stages from the patient's initial tooth arrangement to the target final tooth arrangement, where a separate 3D model is associated with each treatment stage.

Once an adequate set of 3D models is generated, the 3D models may be saved to the patient profile. The dental practitioner may then navigate to a delivery mode to electronically send the completed patient profile to a processing center. The processing center may then generate the custom made series of clear aligners for the patient and deliver the clear aligners to the dental practitioner. The patient would then return to the dental practitioner to receive the first set of clear aligners and verify the clear aligners properly fit onto the patient's teeth.

In one embodiment, a doctor may erase or remove a portion of the 3D model of the dental arch that the doctor has determined to have a low quality. Medical application 115 may direct a user to generate one or more additional intraoral images of the dental site corresponding to the portion of the 3D model (and/or corresponding set or sets of intraoral scans) that was deleted or removed. The user may then use the scanner 150 to generate the one or more additional intraoral scans, which at least partially overlaps with previously generated intraoral scans. The one or more additional intraoral scans may be registered with the 3D model (and/or with the intraoral scans data sets used to create the 3D model) to provide a composite of the 3D model and the one or more additional intraoral scans. In the composite, the part of the 3D model that was previously deleted/removed is at least partially replaced with a corresponding part of the one or more additional intraoral scans. However, the portions of the one or more additional scans that are outside of the deleted or removed part of the 3D model may not be applied to the composite or updated 3D model.

Navigation or control of the user interface of the medical application 115 may be performed via user input. The user input may be performed through various devices, such as a touch input device (e.g., a touchscreen), keyboard, mouse, or other similar control devices. User input may also be provided via scanner 150 in embodiments, such as via a touchpad and/or touchscreen of the intraoral scanner 150. Navigation of the user interface may involve, for example, navigating between various modules or modes, navigating between various segments, controlling the viewing of the 3D rendering, or any other user interface navigation.

A novice user of medical application 115 may not know how to use medical application 115, how to navigate between menus and modes of medical application 115, and so on. Accordingly, in embodiments both local computing device 105 and another user device (e.g., scanner 150, smart speaker 175, IoT pin 180, mobile phone 151, tablet computer 152, laptop computer 154, display 156, etc.) are connected to remote server computing device 106 to gain access to medical virtual assistant 109. A user may provide a prompt (e.g., a voice prompt or text prompt) asking medical virtual assistant 109 how to perform an operation on medical application 115, how to navigate medical application 115, and so on. Responsive to such an prompt, chat model 114, ML model(s) 116 and/or search engine 122 may operate to determine what the user wants and what instructions to issue to medical application 115 to achieve what the user wants. The medical virtual assistant 109 may then send instructions to the local computing device 105 to cause the local computing device to perform the actions it was determined that the user wanted to be achieved. In some embodiments, rather than or in addition to executing the determined operations, the instructions sent to local computing device 105 cause the medical application 115 to display graphics showing how to perform the actions that will achieve the user's desired result, thus teaching the user how to use medical application 115.

Medical application 115 may be complex software that provides clinical treatment information, that plans clinical treatment, that manages clinical treatment, and so on. There can be a steep learning curve to learning use of the medical application 115. Accordingly, in embodiments the medical virtual assistant 109 may answer questions about use and operation of medical application 115, and may even control operation of medical application 115 based on user instructions delivered to the virtual medical assistant via a verbal prompt and/or text prompt. For example, a user may speak into any of the user devices (e.g., IoT pin 180, smart speaker 175, mobile phone 151, etc.) with a request to perform an operation on medical application 115 or to show the user how to perform the operation on the medical application 115. Responsive to such a query, the chat model 114, ML model(s) 116 and/or search engine 122 may work together to generate a response to the prompt, where the response may include control instructions for the medical application 115. The remote server computing device(s) 106 may be connected to local computing device 105, and may send instructions for performing operations on medical application 115 to local computing device 105. Such instructions may then be executed by medical application 115 to achieve the requested outcome (e.g., to zoom in on a region of a presented 3D model, to change modes on the medical application, and so on.

In embodiments, a user may make a verbal request into one of the user devices that lacks a display to select another user device (e.g., display 156) to output a display of intraoral scan data and/or a generated 3D surface to. The verbal request may be a prompt to medical virtual assistant 109, which may process the prompt to determine which of the user devices (e.g., display 156) the user intended to stream the intraoral scan data and/or generated 3D surface to. The medical virtual assistant 109 may then send instructions to local computing device 105 and/or the determined user device (e.g., display 156) to cause the two devices to become paired. After the pairing, the view of the 3D surface and/or intraoral scan data may be streamed to the additional user device (e.g., display 156).

In embodiments, each of the user devices of dental office 108 and remote server computing devices 106 implement a security layer to secure communications between the user devices and the remote server computing devices 106. For example, the user devices and server computing devices 106 may implement WS02 authentication. The security layer may encrypt messages of a sender (e.g., user device or server computing device) and decrypt messages of a receiver (e.g., user device or server computing device) to ensure a secure encrypted communication.

In one embodiment, each of the user devices of dental office 180 that are configured to interface with medical virtual assistant 109 includes credentials for accessing remote server computing device(s) 106 and/or patient records stored at remote server computing device(s) 106. The credentials may be used to identify and authenticate the dentist office 108 and/or the doctor and/or staff of the dental office 108. The identity of the authenticated dental office, doctor and/or staff may be used as context information that affects what information is returned to a user by medical virtual assistant. For example, different user devices may be associated with different users. A doctor may have access to information that his or her staff does not have access to. If a user does not have access to certain treatment context information, then such treatment context information may not be used in formulating a response to a prompt by that user.

In one embodiment, each user device (e.g., scanner 150, local computing device 105, etc.) and/or account into which a user device may be logged may be associated with a particular dental office and/or individual, and the ownership information may be registered with back-end treatment context support system 110. Remote server computing device 106 may use such information to determine how to formulate a response to a user's query in embodiments.

In some instances, medical virtual assistant 109 may formulate a response to a prompt that is not presentable on a user device from which a prompt was generated. For example, a response may include a message that includes text and/or images, but the prompt may have been received from IoT pin 180 or smart speaker 175, neither of which includes a display. In such instances, medical virtual assistant 109 may generate a message that includes a response to the prompt, and send the message to another device of a same user of the user device from which the prompt was received and/or to an account (e.g., email, social media account, etc.) of that user. Additionally, medical virtual assistant may display a notification on the user device from which the prompt was received to alert the user that a message is waiting for them. In an example, the user device may include one or more lights that may light up, flash, etc. to alert the user that action is required or that a message is waiting for them. In the case of a user device that includes an application configured to interface with the medical virtual assistant 109, notifications may be exposed through a notification system of the application and/or of the underlying user device. In the case that the user device from which the prompt is received is intraoral scanner 150, notifications may appear on a display of the intraoral scanner 150 and/or on a display of local computing device 105 that is connected to intraoral scanner 150.

In embodiments, medical virtual assistant 109 includes a chat model 114, one or more ML models (e.g., LLMs) 116 and/or a search engine 122. Chat model 114 may be configured to receive prompts, and to provide those prompts to one or more ML models 116 and/or search engine 122. Chat model 114 may additionally be configured to receive responses to prompts from the one or more ML models 116, and to generate and send search queries to the search engine 122 based on the received responses in embodiments.

Chat model 114 may further be configured to perform one or more actions, which may include actions (e.g., such as scheduling tasks) and/or updates in one or more other systems, such as an intraoral scanning system, a treatment planning system, a treatment management system, a practice management system, and so on. In embodiments, chat model 114 comprises an agent framework capable of orchestration of tasks across multiple systems.

In some embodiments, chat model 114 is an ML-based chat model that uses natural language processing (NLP) techniques to understand and generate human language. This involves several key tasks, including tokenization (breaking down sentences into words or tokens), parsing (analyzing the grammatical structure of sentences), named entity recognition (identifying proper nouns, such as names of people, places, or organizations), and optionally sentiment analysis (determining the emotional tone of a conversation).

In some embodiments, chat model 114 is a rule-based model that uses a set of predefined rules to determine how to respond to user inputs. Such rules may be written, for example, in the form of “if-then” statements. In some embodiments, chat model 114 includes one or more decision trees that guide the chat model 144 to perform one or more actions, such as sending prompts to ML model(s) 116, sending responses from ML model(s) 116 to search engine 122 and/or to a user device, sending instructions to local computing device 105, and so on. A decision tree is a flowchart-like structure where each node represents a user input, and the branches represent possible responses. The chat model may follow the tree based on received input (e.g., from a user device, from an ML model 116, etc.), moving from one node to the next until it reaches a leaf node, which provides a final answer or action.

In embodiments, chat model 114 acts as an intermediary between one or more ML models 116, user devices, medical applications (e.g., medical application 115), practice management systems, and/or search engine 122. Chat model 114 may receive a prompt or query from a user device and forward the prompt to one or more ML models 106A-B. In embodiments, the one or more ML models 116 include a first LLM 116A and a second LLM 116B. The first LLM 116A may be trained to identify clues pertaining to treatment context from a received prompt and to generate one or more search queries based on the identified clues. In embodiments, first LLM 116A is a special instance of an LLM that was trained on customer data, medical services data, medical product data, patient historical data, etc. associated with a provider of medical services and/or medical products. In some embodiments, first LLM 116A is pretrained from knowledge of a particular medical domain. For information that is dynamically updated (e.g., because it is treatment specific, involves new data, etc.), a retrieval augmented generation (RAG) implementation may be implemented. The first LLM 116A may send the generated search query or queries back to chat model 114, which may then send the search query or queries to search engine 122. In embodiments, the generated search query is optimized for search engine 122.

In some embodiments, search engine 122 is a keyword search engine that retrieves information based on matching keywords or phrases (e.g., as output by the first LLM 116A) with the content indexed in its database. A keyword search engine primarily relies on the presence of exact words or phrases in the search query to find and rank relevant results. A keyword search engine may work by building an index of contents (e.g., clinical context and/or treatment context information from data store(s) 130), which may be a structured database containing all the words and phrases found in the content. When a keyword search engine receives a search query, the search engine breaks it down into individual keywords. It then searches its index for information/contents that contain these exact keywords. The search engine may then rank results based on several factors, such as keyword frequency, keyword placement, information priority ratings, and so on. In some embodiments, a keyword search engine supports Boolean operators (e.g., AND, OR, and NOT), that may be used to refine a search.

In some embodiments, search engine 122 is a cognitive search engine. A cognitive search engine is an advanced search platform that goes beyond traditional keyword-based search by incorporating artificial intelligence (AI) and machine learning (ML) technologies to understand and interpret the intent and context of a user's query. Cognitive search engines aim to deliver more relevant, personalized, and insightful search results by leveraging various AI capabilities such as natural language processing (NLP), semantic understanding, and knowledge graphs. Cognitive search engines can understand and process natural language queries, allowing users to search in a more conversational or intuitive manner. This means the engine can interpret questions or complex phrases rather than just matching keywords. Instead of relying solely on keyword matching, cognitive search engines understand the meaning behind words and phrases. They can identify synonyms, related concepts, and the context in which terms are used, providing results that are more aligned with the user's intent. Cognitive search engines take into account the context of a query, which may include the user's original prompt, prior user prompts, etc. in addition to the search query outputs by the first LLM 116A to deliver more accurate results. Cognitive search engines can also maintain context across a series of queries, refining results based on the ongoing conversation. Cognitive search engines may use machine learning models to rank search results based on their relevance, taking into account various factors like user behavior, past interactions, and content quality. These models continuously learn and adapt to improve the accuracy of search results over time. Cognitive search engines often use entity recognition to identify and link specific entities (such as people, places, organizations) in a query. They also leverage knowledge graphs, which are structured databases of interconnected information, to provide enriched search results that include relationships between different concepts. Some cognitive search engines can handle different types of data inputs, such as text, images, video, and even voice. For example, a user might upload an image and receive search results related to the content of that image. Beyond retrieving contents, cognitive search engines can analyze and summarize information, providing insights or actionable knowledge directly in the search results. This could include extracting key points, generating summaries, or even predicting trends based on the data. For example, a cognitive search engine may quickly retrieve relevant medical research, patient records, and/or treatment options from data store(s) 130.

Search engine 122 may perform searches on data store(s) 130 based on one or more search queries provided by the one or more ML model(s) 166 (e.g., first LLM 116A). The data stores 130 may be or include databases, file systems, key-value storage systems, and/or other types of data stores. The data stores 130 may include historical patient information 135 such as patient records that include before treatment and after treatment information, including images, 3D models, doctor notes, and so on. Data store(s) 130 may additionally include current patient information 138 for current patients for which treatment is being performed or might be performed. Such current patient information 138 may include, for example, patient case details, current images, past images, current 3D models (e.g., of dental arches), past 3D models, patient health conditions, patient age, patient gender, patient name, and so on. Data store(s) 130 may additionally include medical product information 140 for one or more medical product suppliers. For example, medical product information 140 may include details of orthodontic aligner products, intraoral scanners, dental computer aided drafting and computer aided manufacturing (CAD/CAM) software, retainers, orthodontic treatment planning software, and so on. Medical application information 142 may include information on one or more medical applications (e.g., such as medical application 115), including how the medical applications work, application programming interfaces (APIs) of the medical applications, function calls and instructions that can be made to the medical applications to control those medical applications, and so on.

Search engine 122 may provide search results (e.g., which may include treatment context information) to chat model 114. In some embodiments, chat model 114 may provide the search results, the search queries that triggered the search results and/or the initial prompt that caused the first LLM 116A to generate the search query back to the first LLM 116A. The first LLM 116A may then generate one or more new search queries, which chat model 114 may feed back into search engine 122. The search engine 122 may generate updated search results. One or more rounds of this process may be performed until the first LLM 116A determines that it has acquired all of the treatment context information that is needed and/or that it is able to obtain.

Chat model 114 may provide the initial prompt, a chat history (e.g., back and forth between a user and the chat model 114), treatment context information obtained from one or more iterations of searches and results (e.g., generated based on operations of the first LLM 116A and the search engine 122) and/or additional instructions to a second ML model 116B (e.g., which may be a second LLM). In embodiments, second LLM 116B is a special instance of an LLM that was trained on customer data, medical services data, medical product data, patient historical data, etc. associated with a provider of medical services and/or medical products. In some embodiments, second LLM 116B is pretrained from knowledge of a particular medical domain. For information that is dynamically updated (e.g., because it is treatment specific, involves new data, etc.), a retrieval augmented generation (RAG) implementation may be implemented.

The second LLM 116B may then generate a response to the user's initial one or more prompts that is informed by the treatment context and provide the response to chat model 114. Chat model 114 may then send the response to the user device that initiated the interaction, may send a message to one or more other devices, may send instructions to control an operation of medical application 115 to local computing device 105, and so on.

In embodiments, one or more of the ML model(s) 116, chat model 114 and/or search engine 122 that make up medical virtual assistant 109 are custom designed large language models and/or supporting systems that support many use cases. In a first example, when asked by a user, the medical virtual assistant 109 will provide specific answers about products & services of a provider (e.g., such as Align Technology®). In a second example, when asked by a user, the medical virtual assistant 109 will provide specific answers about publicly available data published about particular types of medical treatments, such as clear aligner therapy. These answers may include solutions specific to a particular medical product/service provider and their clinical applications in embodiments. In a third example, when asked by a user, the medical virtual assistant 109 will provide specific answers to orthodontic questions. In a fourth example, when asked by a user, the medical virtual assistant 109 will provide specific answers to dental questions. In a fifth example, medical virtual assistant 109 provides specific answers to account questions for accounts with a particular medical product/service provider. In a sixth example, medical virtual assistant 109 provides historical patient information for patients having specific properties. For example, a doctor may ask for images of patients having particular characteristics, and medical virtual assistant 109 may retrieve and provide such images. In a seventh example, medical virtual assistant 109 may answer questions about how to navigate or use a medical application, and may show and/or describe steps for doing so. For example, a doctor may ask to be shown the steps for a treatment plan in which one or more teeth are extracted vs. a treatment plan where no teeth are extracted. In response, medical virtual assistant 109 may determine the steps for both types of treatment plan, and show both while comparing and contrasting the two. In another example, medical virtual assistant may take control of a mouse pointer in a medical application, causing the mouse pointer to click on an icon or sequence of icons to implement requested functionality of the medical application. Such actions may be shown with an overlay explaining the actions.

In embodiments, additional functionality provided by medical virtual assistant 109 are live chat and chatbot functionalities through voice input, an ability for customers to call appropriate personnel at a medical product/service provider, an ability for medical practices to use user devices to communicate with each other (e.g., where medical virtual assistant 109 functions as an inter-office intercom), and an ability for practices at different locations to communicate with each other quickly through user devices.

Each user device in dental office 108, one or more other dental offices, a laboratory (lab), a manufacturing facility, etc. may be associated with a particular user. For example, each user may include their own IoT pin that is associated with that user. In an example, a user using a first user device may request to connect with a second user of a medical practice, of a lab that a medical practice is partnering with to design and/or prepare a medical product (e.g., orthodontic aligners), of a medical product/service provider, etc. Medical virtual assistant 109 may determine a user device used by the second user (e.g., based on a search query made to search engine 122), and may then act as a go-between between those two devices. For a conversation between the two devices, what one person says may be sent to medical virtual assistant, which may then forward that information (e.g., audio and/or text) to the user device of the other user, enabling a conversation to take place. In an example, a doctor may use a particular lab to prepare one or more dental prosthetics for their patients, and may have questions for the lab and/or instructions for the lab. The doctor may ask medical virtual assistant to 109 to connect the doctor to the lab, and in response medical virtual assistant 109 may determine a contact (e.g., a technician) at the lab that is working on the project for the doctor and initiate and manage a connection between the doctor and that contact as discussed above.

In some embodiments, medical virtual assistance 109 may create coherent soundscapes across doctor office 108 by coordinating music/sound between user devices through integrated music services (e.g., such as Spotify, Pandora, Apple Music, etc.).

In some embodiments, a doctor or staff of dental office 108 may provide instructions to medical virtual assistance 109 via one of the user devices to create a patient record, to add information to a patient record, to schedule visits for a patient, to begin development of a treatment plan for the patient, and so on. For example, the medical virtual assistant 109 may integrate with a practice management system of dental office 108 to allow staff to update patient information in real-time through voice prompts. This may include, for example, updating patient records by saying, “Patient Status: Sam Brown Missed Appointment”, “Patient Update: Sam Brown in Operator 2”, or the like. Additionally, the medical virtual assistant 109 may enable saving/entering a prescription for a patient without requiring the doctor to type in the prescription. For example, the doctor may speak the prescription into the user device, and the medical virtual assistant 109 may understand the prescription and record it in the medical record of the patient and/or send the prescription to a pharmacy and/or lab. The user may not need to provide specific scripted instructions for medical virtual assistant 109 to understand what the user is attempting to accomplish in embodiments.

In some embodiments, a doctor or staff may ask questions of the medical virtual assistant 109 that are about a treatment plan that has been generated (e.g., by treatment planning software and/or a lab technician). Examples of questions and/or prompts that may be asked by the doctor include, “why was this type of attachment added?”, “why this type of staging?”, “provide educational information from education tab,” “provide clinical information for treatment,” and so on. A doctor or staff may also ask questions about available or recommended practice management and support tools, about an order status of a medical product (e.g., of a prosthodontic or orthodontic aligner), and so on. In each instance, the medical virtual assistant 109 may determine an answer to the question/prompt and provide the answer to the user.

In embodiments, virtual medical assistant 109 determines contextual clues about the environment from which a prompt/request is being asked, and formulates a response in view of the determined environment. For example, virtual medical assistant may determine whether a doctor is with a patient or not with a patient (e.g., based on image data showing a patient, audio data identifying a patient, and so on. The virtual medical assistant may formulate answers differently based on whether or not the patient is present. For example, the virtual medical assistant may send a response to a device or medium that can be viewed and/or heard by the doctor but not by the patient. If a response will be delivered in earshot of the patient, then the response may be tailored in such a way to not cast doubt on the doctor's expertise. In another example, medical virtual assistant 109 may determine a level of privacy surrounding a received prompt, and may limit or tailor a response based at least in part on the determined level of privacy. For example, if the doctor is in a public setting then the response may not include confidential patient information that might be heard and/or viewed by the public.

Virtual medical assistant 109 may be “context-aware”, in that it prepares responses to prompts in view of contextual information that may be determined by one or more aspects or components of the virtual medical assistant 109, such as by the one or more ML models 116A-B, the chat model 114, and/or the search engine 122. The virtual medical assistant 109 may gather treatment context for a doctor, evaluate the treatment context for actionable recommendations based on the treatment context, and provide the actionable recommendations to the doctor. The treatment context may include one or more of a clinical context, a patient-doctor interaction context (e.g., is the patient in the same room as the doctor) and/or a practice management context in embodiments. The virtual medical assistant 109 may receive audio data, motion data and/or image data, and may use any one or more of such data to formulate a response to a prompt in embodiments. Actionable recommendations may include treatment information, digital practice management information, and/or doctor-patient relationship information. Actionable recommendations may be provided, for example, through audio, visual and/or haptic feedback.

In embodiments, medical virtual assistant 109 closely ties to a digital dental ecosystem of a medical product/service supplier, such as Align Technology. Medical virtual assistant 109 may be able to access and leverage information about specific doctors and their preferences, about specific patients (e.g., including medical history, personal information, insurance information, current and past prescriptions, diagnostics data for the patients, 3D models of dental arches for the patients, treatment plans for the patients, and so on). To access such information, the medical virtual assistant 109 may tie into a practice management system, a treatment planning system, an intraoral scanning system, a treatment management system, a virtual care system, and so on.

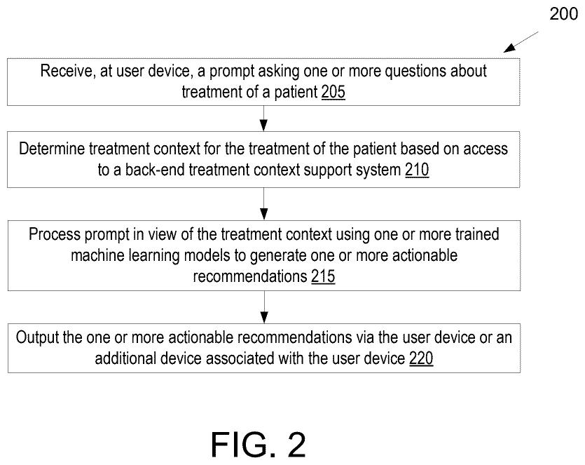

FIGS. 2-5 illustrate methods and a sequence diagram related to a medical virtual assistant. Operations of the methods may be performed by a processing logic that may comprise hardware (e.g., circuitry, dedicated logic, programmable logic, microcode, etc.), software (e.g., instructions run on a processing device to perform hardware simulation), or a combination thereof. In one embodiment, at least some operations of the methods are performed by processing logic of a remote server computing device, which may include processing logic for one or more ML models 116, a chat model 114 and/or search engine 122 of FIG. 1.

For simplicity of explanation, the methods are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

FIG. 2 is a flow diagram for a method 200 of providing a medical virtual assistant, in accordance with an embodiment. At block 205 of method 200, a user device (e.g., a wearable IoT device, a smart speaker, a mobile phone running a medical voice assistant application, etc.) receives a prompt associated with treatment of a patient. The prompt may be a prompt asking questions about a status of the patient, asking for medical records and/or conditions of the patient, asking for help in operating a medical application that has a file open for the patient, asking a status of an order for the patient, and so on. The prompt may then be transmitted from the user device to a server computing device associated with a medical virtual assistant.

At block 210, processing logic of the server computing device may process the prompt and/or additional information to determine a treatment context (e.g., a clinical context) for the patient based on access to a back-end treatment context support system. The additional information may include audio data received with the prompt, such as background noise that may provide clues as to whether the user of the user device who generated the prompt is with a patient, is in a doctor office, is out in public, is currently using a medical application, and so on. In some embodiments, the additional information may include prior information received from the user in a current chat session, such as a full chat history with the user. In some embodiments, the additional information is received from an intraoral scanning application, a treatment planning application and/or a treatment monitoring application that is in use by the user of the user device.

In embodiments, the prompt and/or additional information may be provided to an artificial intelligence model such as an LLM configured to generate search queries in a treatment context and/or clinical context data store. The artificial intelligence model may process the prompt and/or additional information to generate a query to be executed by a search engine, which may itself be another artificial intelligence model in embodiments. In an example, the prompt or additional information may include a name of a patient, and the artificial intelligence model may generate a search query for medical information about that patient. The search engine may then run the search query for the medical information about that patient, and may receive medical records of the patient. In some embodiments, the search engine may receive information for the treatment context of the patient and provide the treatment context as well as the prompt and/or additional information to the artificial intelligence model or another artificial intelligence model (e.g., a second artificial intelligence model such as a second LLM 116B).

In embodiments, the LLM receives one or more clues for the treatment context (e.g., clinical context) via the user device and/or an associated device. The clues may include an image of a patient, which may be processed with a machine learning model to perform face recognition and determine an identify of the patient, a name of the patient spoken by the user, captured speech of the patient, intraoral scan data of a dentition of the patient, additional information about the patient spoken by the user, and so on. The clues may additionally include information on a medical application that is running and that has an active user account corresponding to a user that generated the prompt. In embodiments, processing logic performs a lookup in a data store of a back-end treatment context support system for information on the treatment context and/or clinical context based on the one or more clues. For example, the one or more clues may be used to formulate a search query. A result of the search query may include an identity of the patient, patient records (e.g., patient information of the patient), historical records on other patients with similar medical conditions, information on a medical application under use, and/or many other types of information, which may be retrieved from the data store in embodiments.

In some embodiments, clinical context information may include, for example, a patient identity, one or more procedures and/or treatments being performed on the patient, one or more prior procedures and/or treatments previously performed on the patient, patient history, and so on. In some embodiments, the user device and/or another device (e.g., an intraoral scanner) captures image data of the patient. Additionally, or alternatively, the user device or another user device may capture audio data of the patient. Such information can be processed to determine an identity of the patient in embodiments. For example, a 3D surface may be generated from intraoral scans, and may be used to search a data store of 3D models of dental arches/jaws of patients to find a match. Responsive to finding a match, a known patient associated with the match may be identified. In embodiments, the medical virtual assistant 109 may identify a current patient that the doctor is working on/seeing without the doctor explicitly informing the medical virtual assistant 109 of an identity of the patient. Then, any questions/prompts addressed to the medical virtual assistant 109 may be answered with respect to the determined patient at hand.

In some embodiments, the user device and/or another device captures one or more images indicating a presence or lack of presence of the patient. Additionally, or alternatively, the user device or another user device may capture audio indicating a presence or lack of presence of the patient. The presence or lack of presence of the patient may be one piece of contextual information that the one or more LLMs may use to formulate actionable recommendations. In embodiments, the content of the actionable recommendations is based at least in part on the presence or lack of presence of the patient near the doctor or staff that issued the prompt. For example, if the patient is with the doctor, then the actionable recommendation might not explain treatments, conditions, or medical application uses in a manner that could make the patient doubt that the doctor knows such information.

In one embodiment, the user device is an intraoral scanner, and the one or more clues for treatment context include image data (e.g., scan data) captured by the intraoral scanner, movement data of the intraoral scanner, and/or data indicating that the intraoral scanner is currently in use. Such information indicates that the doctor is in the presence of the patient, and may be used to tailor a response to the prompt from the doctor.

At block 215, processing logic processes the original prompt, additional information, and/or treatment context (e.g., may process the prompt in view of the treatment context) using one or more trained artificial intelligence models (e.g., the first LLM or a second LLM) to generate one or more actionable recommendations. The nature of the actionable recommendations will be highly dependent on the initial prompt that was provided. If the prompt was a request for patient information, then the response may include the patient information. If the prompt was for an assessment of the patient's health conditions (e.g., of caries, tooth wear, gum disease, periodontal disease, etc.), then the response may include an automated assessment of the patient's health conditions processed using one or more health diagnostics tools. If the prompt was a request about status of an order for the patient, then the response may include an order status. If the prompt was a request about why a treatment plan for a patient includes a particular treatment option (e.g., a particular type or placement of an attachment on a patient tooth, a particular staging of orthodontic treatment, a particular amount of movement for one or more teeth, etc.), then the response may include an explanation of why the particular treatment option was included in the treatment plan. If the request was for prior patients who had experienced similar health issues (e.g., had similar malocclusions) as the patient, then the response may include a list of such prior patients, optionally along with associated images and/or 3D models of the pre-treatment and/or post-treatment dentition for such prior patients. If the prompt was an instruction to perform an action (e.g., schedule a patient visit, issue a prescription, control a medical application, etc.), then the action may be performed. Many other use cases are also envisioned.

At block 220, the server computing device outputs the one or more actionable recommendations to the user device. The user device may then output the one or more actionable recommendations. Additionally, or alternatively, the one or more actionable recommendations may be sent to an additional device associated with the user. For example, if the user device was an IoT pin, such a user device may lack a monitor capable of displaying images, 3D models, etc. Accordingly, processing logic may determine a different user device or medium of the user to send the actionable recommendation(s) to. In such an instance, a notification may be sent to the user device that the actionable recommendation(s) are available for review on the additional user device. The notification may include, for example, a flashing light, an audio indicator, a voice message telling the user to check their email/social media/other user device, etc.

In embodiments, actionable recommendations may include treatment information, digital practice management information and/or doctor-patient relationship information. Treatment information may include information about a treatment (e.g., an orthodontic treatment, prosthodontic treatment, etc.) of a patient at hand, such as what operations are included in the treatment, a type of orthodontic appliance to use (e.g., braces, clear aligners, retainers, etc.), staging of the treatment, estimated treatment duration, specific treatment goals (e.g., alignment, bite correction, spacing), recommended interproximal reduction (IPR), tooth extractions and/or other pre-treatment or intra-treatment procedures, tooth attachments, amount of tooth movement at different stages, occlusal contact information, and so on. In the context of prosthodontics, treatment information might include a type of prosthesis (e.g., crown, bridge, dentures, implants, etc.), material choices (e.g., porcelain, metal, resin, etc.), step-by-step procedure details (e.g., tooth preparation, impressing taking, temporary prosthesis), coordination with other dental or medical treatments (e.g., periodontal therapy, surgery, etc.), and so on. Treatment information may additionally or alternatively include information on each procedure involved, including taking of impressions, x-rays, tooth preparation, appliance fittings, adjustments, and so on, frequency of visits, and/or whether sedation or anesthesia is to be used and how much. Treatment information may include expected outcomes such as aesthetic goals (e.g., expected changes in appearance, such as smile design, tooth alignment and facial symmetry), functional goals (e.g., improvement in bite, speech, chewing, and overall dental function), and/or any post-treatment care (e.g., including retainers, hygiene recommendations, follow-up visits, etc.). Treatment information may include a breakdown of costs, insurance coverage information, payment plans, etc. Treatment information may include pre-treatment patient instructions, post-treatment care, emergency protocols, etc. Treatment information may include before and after images, 3D models, radiographs, intraoral scans, etc. Treatment information may include progress notes such as detailed records of each patient visit including what was done, patient responses, and any modifications to the treatment plan.

Digital practice management information in dentistry or orthodontics typically includes a range of details that help to streamline administrative, clinical, and financial aspects of a dental practice. Actionable recommendations may include information about and/or control of any type of digital practice management information. Digital practice management information may include patient records (e.g., including demographics, medical history, dental history, treatment plans, etc.). Digital practice management information may include appointment scheduling information, such as calendar management, real-time availability, appointment types, and automated reminders. In an example, a user may issue a prompt asking for free time windows to schedule a patient appointment, responsive to which the medical virtual assistant may provide such open time windows. The doctor or staff may then select one of the windows via voice command, which may prompt the medical virtual assistant to schedule an appointment for that time window and update a practice management system by entering the appointment into the practice management system.

Digital practice management information may include billing and financial management information. This includes generation of invoices, payment tracking, insurance claim handling, setting up and tracking of payment plans, generation of financial reports, and so on. Any such actions may be initiated using the medical virtual assistant, and inquiries may be made about any such information via the medical virtual assistant.

Digital practice management information may include management of electronic health records, such as clinical notes (e.g., documentation of each patient visit, including what procedures were performed, observations, and recommendations), treatment progress (e.g., tracking of ongoing treatments with timelines, milestones, and outcomes), digital imaging (e.g., storage and access to digital x-rays, 3D scans, intraoral photos, and other diagnostic images), prescriptions (e.g., including refill tracking), and/or referrals (e.g., documentation and tracking of referrals to specialists or other healthcare providers). Any such actions may be initiated using the medical virtual assistant, and inquiries may be made about any such information via the medical virtual assistant.

Digital practice management information may include inventory and supply management, such as inventory tracking (e.g., monitoring of dental supplies, orthodontic appliances and prosthodontic materials), automated reordering (e.g., triggering reorders when inventory levels fall below a certain threshold), and/or supplier management (e.g., tracking orders, deliveries and supplier performance). Any such actions may be initiated using the medical virtual assistant, and inquiries may be made about any such information via the medical virtual assistant.

Digital practice management information may include compliance and security, such as Health Insurance Portability and Accountability Act (HIPAA) compliance (e.g., ensuring that patient data is handled in accordance with health privacy regulations), data security (e.g., encryption of patient records, secure data storage, access control, etc.), audit trails (e.g., logs of all access to patient records and actions taken), and consent forms (e.g., digital management of consent forms for treatments, allowing for electronic signatures and storage). Any such actions may be initiated using the medical virtual assistant, and inquiries may be made about any such information via the medical virtual assistant.

Digital practice management information may include information on reporting and analytics, such as practice performance (e.g., analysis of patient volume, treatment outcomes, financial performance, appointment trends, etc.), patient demographics, treatment analytics (e.g., tracking the success rates and satisfaction with various treatments), operational efficiency (e.g., monitoring of appointment durations, staff productivity, resource utilization, etc.), and so on. Any such actions may be initiated using the medical virtual assistant, and inquiries may be made about any such information via the medical virtual assistant.

Digital practice management information may include staff and provider management, such as management of provider schedules (e.g., including availability, vacations, shift assignments, etc.), credentialing (e.g., keeping records of provider credentials, certifications, and continuing education), performance reviews, and task management. Any such actions may be initiated using the medical virtual assistant, and inquiries may be made about any such information via the medical virtual assistant.

Digital practice management information may include marketing and patient acquisition, such as patient referrals (e.g., tracking referral sources and effectiveness), marketing campaigns (e.g., management of email campaigns, social media efforts, and promotions), and new patient onboarding (e.g., streamlining the process for new patients, including digital forms and initial consultation). Any such actions may be initiated using the medical virtual assistant, and inquiries may be made about any such information via the medical virtual assistant.

Doctor-patient relationship information may include patient background and preferences, such as personal preferences (e.g., information about the patient's preferences regarding treatment approaches, comfort levels, and any specific requests such as preference for certain types of anesthesia or pain management), cultural and religious considerations (e.g., understanding of any cultural, religious, or personal beliefs that may impact treatment choices or communication style), communication preferences (e.g., preferred method of communication such as email, phone or text, language preferences, and frequency of updates), and/or previous experiences (e.g., insights into the patient's past experiences with dental or orthodontic treatments, including anxieties, fears, or positive experiences). Any such actions may be initiated using the medical virtual assistant, and inquiries may be made about any such information via the medical virtual assistant.

Doctor-patient relationship information may include trust and rapport-building efforts, such as initial consultations (e.g., details about how the doctor introduced themselves, discussed treatment options, and addressed patient concerns), ongoing communication (e.g., notes on how the doctor maintains regular communication with the patient, provides updates on treatment progress, and responds to inquiries or concerns), and/or empathy and understanding (e.g., documentation of the doctor's efforts to understand the patient's emotional and psychological state, particularly regarding anxiety related to dental procedures). Any such actions may be initiated using the medical virtual assistant, and inquiries may be made about any such information via the medical virtual assistant.

Doctor-patient relationship information may also include informed consent details, such as detailed explanations (e.g., records of how the doctor explained treatment options, risks, benefits, and alternatives to the patient in a way that was understandable and thorough), patient questions (e.g., notes on any questions the patient asked and how the doctor addressed them), and/or consent forms (e.g., signed consent forms for specific procedures, indicating that the patient has been fully informed and agrees to the proposed treatment). Any such actions may be initiated using the medical virtual assistant, and inquiries may be made about any such information via the medical virtual assistant.

Doctor-patient relationship information may further include patient education and empowerment, such as educational resources (e.g., information about any brochures, videos, or online resources provided to the patient to help them understand their condition and treatment options), instructions for care (e.g., detailed instructions given to the patient for at-home care, post-procedure care, and ongoing oral hygiene practices), and/or decision-making involvement (e.g., documentation of how the patient was involved in the decision-making process, ensuring they feel empowered to make informed choices about their treatment). Any such actions may be initiated using the medical virtual assistant, and inquiries may be made about any such information via the medical virtual assistant.

Doctor-patient relationship information may include respect for autonomy, such as respect for decisions (e.g., notes on how the doctor respected the patient's decisions, even if they chose a different treatment path or opted to decline a recommended procedure) and/or alternative options (e.g., records of alternative treatment options discussed and any accommodations made based on the patient's preferences or decisions). Any such actions may be initiated using the medical virtual assistant, and inquiries may be made about any such information via the medical virtual assistant.

Doctor-patient relationship information may include confidentiality and privacy measures, such as privacy measures (e.g., documentation of how patient information is kept confidential, including how discussions are held in private settings and how records are securely managed) and/or disclosure agreements (e.g., any agreements regarding the disclosure of patient information, including situations where the patient has given permission for information to be shared with family members or other healthcare providers). Any such actions may be initiated using the medical virtual assistant, and inquiries may be made about any such information via the medical virtual assistant.

Doctor-patient relationship information may further include ethical and professional conduct, such as ethical compliance (e.g., notes on the doctor's adherence to ethical guidelines in patient interactions, ensuring that all recommendations are in the patient's best interest) and/or conflict resolution (e.g., documentation of any conflicts or misunderstandings that arose and how they were resolved in a professional and respectful manner). Any such actions may be initiated using the medical virtual assistant, and inquiries may be made about any such information via the medical virtual assistant.

Doctor-patient relationship information may include follow-up and continuity of care, such as follow-up appointments (e.g., details about how the doctor schedules and conducts follow-up appointments to monitor the patient's progress and address any ongoing concerns) and/or continuity of care (e.g., records showing how the doctor ensures continuity of care, particularly if the patient is referred to a specialist or if multiple providers are involved in the patient's treatment). Any such actions may be initiated using the medical virtual assistant, and inquiries may be made about any such information via the medical virtual assistant.

Doctor-patient relationship information may include patient feedback and satisfaction, such as feedback collection (e.g., information on how patient feedback is collected, such as through surveys or direct conversations, and how that feedback is used to improve the doctor-patient relationship) and/or patient satisfaction (e.g., notes on the patient's satisfaction with their care, including any positive or negative feedback they provided about their interactions with the doctor). Any such actions may be initiated using the medical virtual assistant, and inquiries may be made about any such information via the medical virtual assistant.

Doctor-patient relationship information may include documentation of relationship dynamics, such as interaction records (e.g., detailed records of all interactions between the doctor and the patient, noting any significant changes in the relationship, such as increased trust or emerging concerns) and/or personalized care (e.g., documentation of how the doctor tailors care to the patient's individual needs, preferences, and circumstances, reinforcing a personalized approach to treatment). Any such actions may be initiated using the medical virtual assistant, and inquiries may be made about any such information via the medical virtual assistant.

In one example, the prompt is with regards to a treatment plan for at least one of orthodontic treatment or restorative dental treatment presented by an instance of a medical application. The actionable recommendations comprise at least one of information about an aspect of the treatment plan, an explanation of reasons for the aspect of the treatment plan, or instructions on how to use the medical application.

In embodiments, determining the treatment context at block 210 includes determining an instance of a medical application in use for the patient. In such an embodiments, the one or more trained machine learning models (e.g., LLMs) may further output one or more control instructions for controlling the instance of the medical application. These instructions may be issued to the medical application (e.g., via an API) to control the instance of the medical application. The controlling of the medical application may be performed responsive to the prompt.

In embodiments, the prompt comprises a request for images of prior patients having particular characteristics. In such embodiments, the one or more actionable recommendations may comprise identifiers for one or more pre-treatment images of prior patents having the particular characteristics. The method may further include retrieving the one or more pre-treatment images by the back-end treatment context system, determining one or more post-treatment images associated with the and the one or more pre-treatment images, and outputting the one or more pre-treatment images and the one or more post-treatment images to the additional device for display to the patient.

In some instances, the one or more actionable recommendations may be insufficient to address one or more questions of the prompt. This may be determined, for example, based on determining a probability that the response addresses the questions of the prompt, and determining that the probability is below a probability threshold. Alternatively, processing logic may output a question to the user asking whether the response answered their question. The user may answer in the negative, indicating that the response did not answer their question. Alternatively, the user may provide an updated prompt stating that the response did not answer their question. In such a situation, processing logic may establish a voice connection between the user device and a representative of a company that provides a medical application or a medical product in question. In some embodiments, processing logic selects the representative from a plurality of available representatives based on a determination that the representative will be able to answer the one or more questions. For example, the LLM may process the prompt and/or a further prompt to determine a nature of the inquiry and a department within the company that is associated with the nature of the inquiry. The LLM may further retrieve context information on representatives from that department that are on call and available to talk to the user, determine their phone number or other contact information, and initiate a phone call or other connection to them using the phone call and/or other contact information.

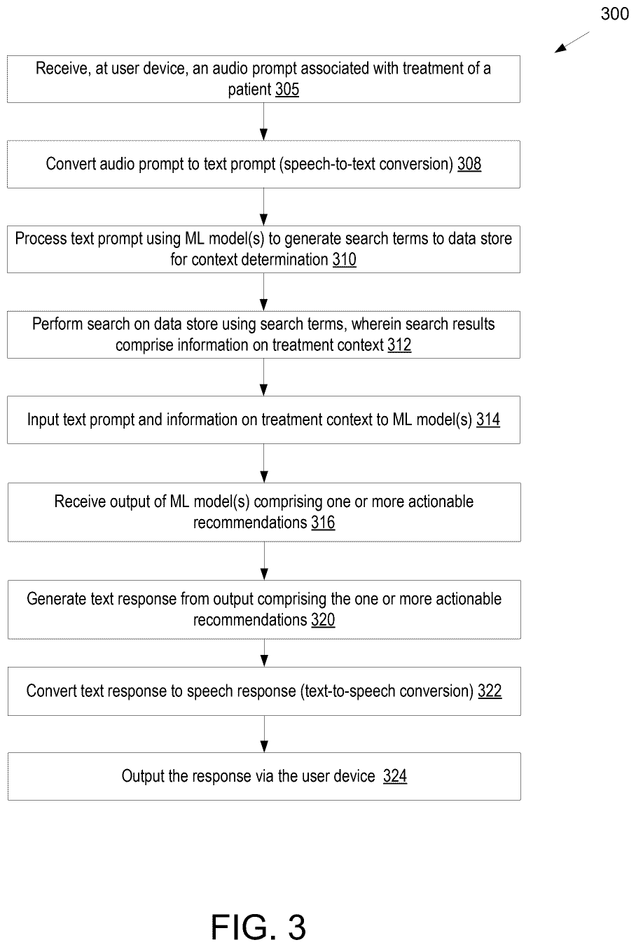

FIG. 3 is a flow diagram for a further method 300 of providing a medical virtual assistant, in accordance with an embodiment. At block 305 of method 300, a user device (e.g., a wearable IoT device, a smart speaker, a mobile phone running a medical voice assistant application, etc.) receives an audio (e.g., voice) prompt associated with treatment of a patient. The prompt may be a prompt asking questions about a status of the patient, asking for medical records and/or conditions of the patient, asking for help in operating a medical application that has a file open for the patient, asking a status of an order for the patient, and so on. The prompt may then be transmitted from the user device to a server computing device associated with a medical virtual assistant.

At block 308, the prompt may be processed to convert the audio prompt into a text prompt via a speech-to-text conversion process.

At block 310, processing logic of the server computing device may process the text prompt and/or additional information using one or more artificial intelligence models (e.g., LLMs) to generate one or more search terms for a search to be run on a data store (or an index of a data store) for context determination. The additional information may include audio data received with the prompt, such as background noise that may provide clues as to whether the user of the user device who generated the prompt is with a patient, is in a doctor office, is out in public, is currently using a medical application, and so on. In some embodiments, the additional information may include prior information received from the user in a current chat session, such as a full chat history with the user.

At block 312, the search terms are input into a search engine, which performs a search of a data store and/or an index of a data store using the search terms. A result of the search may be search results that comprise information on treatment context. In some embodiments, the search engine is a keyword search engine. In some embodiments, the search engine is a cognitive search engine that includes one or more trained artificial intelligence models (e.g., LLMs).

At block 314, processing logic processes the original prompt, additional information, and/or treatment context (e.g., may process the prompt in view of the treatment context) using one or more trained artificial intelligence models (e.g., a second LLM) to generate one or more actionable recommendations.

At block 316, processing logic may receive an output of the artificial intelligence model(s) comprising the one or more actionable recommendations.

At block 320, processing logic generates a text response from the output comprising the one or more actionable recommendations.

At block 322, processing logic converts the text response to a speech response via a text-to-speech conversion process.

At block 324, the server computing device outputs the speech response to the user device. The user device may then output the speech (audio) response via a speaker of the user device.

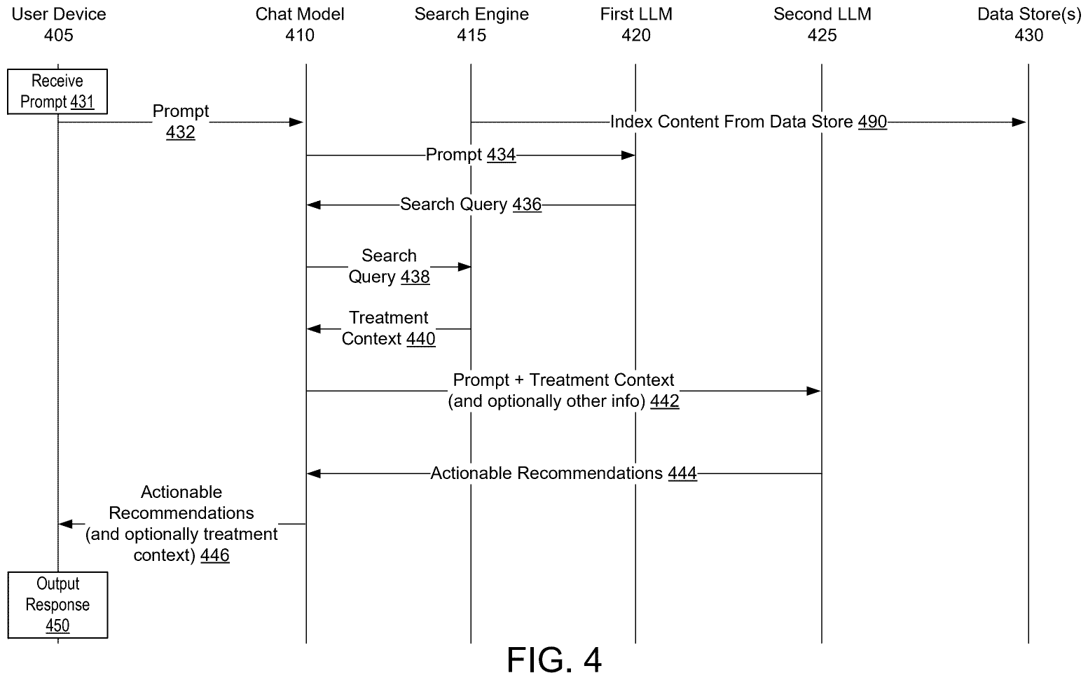

FIG. 4 is a sequence diagram for a method 400 of engaging with a user using a medical virtual assistant, in accordance with an embodiment. The sequence diagram shows a user device 405 (e.g., an IoT device, a mobile phone, a tablet computer, an intraoral scanner, etc.) that interacts with a chat model 410 that provides an interface to a medical virtual assistant. The chat model 410 together with a second LLM 425 may provide the medical virtual assistant. Additionally, a search engine 415, first LLM 420 and data store(s) 430 may together constitute a back-end treatment context support system that the medical virtual assistant may leverage to provide responses that are pertinent to particular patient files, to particular doctors, and so on.

At block 431 of the sequence diagram, user device 405 receives a prompt. The prompt may be a voice prompt received via a microphone of the user device. Alternatively, or additionally, the prompt may be or include a text prompt that was typed into the user device 405. Additionally, or alternatively, the prompt may be or include a gesture prompt that includes motion data recorded by an accelerometer, gyroscope and/or other inertial measurement unit (IMU) of the user device. Additionally, or alternatively, the prompt may be or include a visual prompt that may include a video and/or one or more images generated by the user device.

At block 432, the user device 405 forwards the prompt to the chat model (e.g., to a server computing device hosting the chat model 410. At block 434, the chat model may forward the prompt to first LLM 420. The first LLM may process the prompt to determine clues about a treatment context usable to generate a search query associated with the treatment context. In embodiments, the first LLM transforms the prompt into a search query optimized for search engine 415 (e.g., optimized for a cognitive search engine). The search query may be a query to retrieve treatment context information that was not accessible to first LLM 420 or second LLM 425 at a time of training. The treatment context information may be important in formulating a response to the prompt in embodiments, and an accurate response to the prompt may not be possible without the treatment context in embodiments.

At block 436, the first LLM 420 returns the determined search query 436 to the chat model 410.

At block 438, the chat model 410 inputs the search query 438 to search engine 415. The search engine 415 processes the search query to determine treatment context information, and then at block 440 returns the treatment context information to the chat model 410.

At block 442, the chat model 410 provides the original prompt and the treatment context information (optionally with other information such as a chat history) to second LLM 425.

At block 444, the second LLM processes the original prompt and the treatment context information (and optionally any other provided information) to determine one or more actionable recommendations. At block 410, the chat model provides the actionable recommendations (and optionally the determined treatment context information) to the user device 405. In some embodiments, and/or in response to some prompts, the chat model performs one or more actions, and reports on performance of the one or more actions to the user device 405. At block 450, the user device then outputs a response to the prompt that includes the actionable recommendations and/or the treatment context information.

At block 490, search engine 415 may index the content of the data store(s) 430 to improve an efficiency of search results returned by search engine 415. This indexing process may be performed asynchronously with the other operations of the sequence diagram, and may not be tied to a user interaction. For example, the indexing process may be performed prior to the operations of any of blocks 431-450 in embodiments.

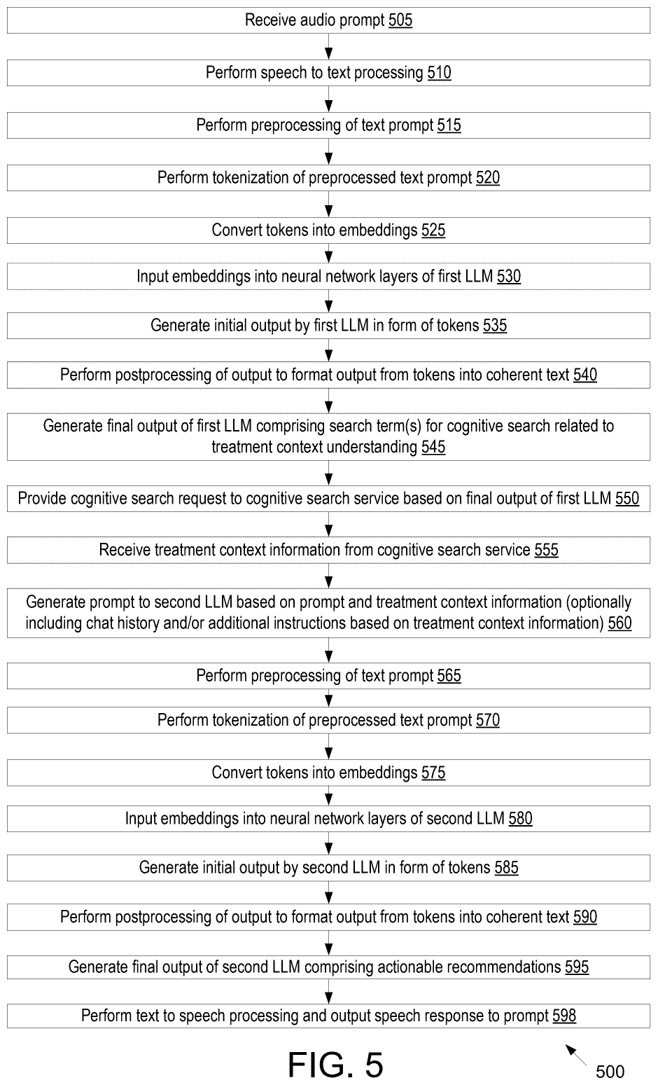

FIG. 5 is a flow diagram for a method 500 of engaging with a user using a medical virtual assistant, in accordance with an embodiment. At block 505 of method 500, a user device (e.g., a wearable IoT device, a smart speaker, a mobile phone running a medical voice assistant application, etc.) receives an audio (e.g., voice) prompt associated with treatment of a patient. The prompt may be captured by a microphone of the user device in embodiments. The prompt may then be transmitted from the user device to a server computing device associated with a medical virtual assistant.

At block 510, the prompt may be processed to convert the audio prompt into a text prompt via a speech-to-text conversion process.

At block 515, processing logic of the server computing device may perform pre-processing of the text prompt to ensure that the input data is clean, standardized, and ready for efficient processing. The pre-processing may include text normalization (e.g., converting all text to lowercase, standardizing or removing punctuation, expanding contractions, etc.), handling of special characters (e.g., stripping out non-alphanumeric characters and/or normalization of accented characters), handling of whitespace (e.g., removing extra spaces, trimming leading and/or trailing spaces, etc.), text cleaning (e.g., removing or replacing URLs, email addresses, and/or other specific tokens, removal of stop words, lemmatization or stemming, etc.), sentence splitting, Unicode normalization, handling os special tokens, and/or other operations.

At block 520, processing logic may perform tokenization of the preprocessed text prompt. Tokenization involves breaking down text into smaller units, called tokens, that a model can process and understand. These tokens are typically the smallest meaningful elements of the text, and the way they are defined can vary depending on the tokenization strategy used. Tokenization can include word-level tokenization, sub-word level tokenization and/or character-level tokenization. Each token may be mapped to a unique integer identifier (ID) based on a vocabulary that the model was trained on. Tokenization may additionally include adding special tokens such as start/end tokens, padding tokens and/or mask tokens. A tokenizer may implement various tokenization strategies, such as greedy tokenization (where the tokenizer chooses the longest possible token from the vocabulary at each step. For example, in the word “playing”, it might select “play” and “ing” if those are the longest matches) and/or detokenization (the reverse process of tokenization, where tokens are combined back into human-readable text).

At block 525, processing logic converts the tokens into embeddings. This step involves mapping each token (usually represented as an integer ID from a vocabulary of the LLM) into a dense vector of real numbers, which the model can then use for further computation. The model may include an embedding layer or embedding matrix that stores embeddings for all the tokens in the vocabulary. This matrix typically has a shape of (vocab_size, embedding_dim), where vocab_size is the total number of unique tokens in the vocabulary, and embedding_dim is the dimensionality of the embeddings (e.g., 128, 256, 512, etc.). For example, if the vocabulary has 10,000 tokens and the embedding size is 300, the embedding matrix would be of size (10,000×300). Each row in the embedding matrix corresponds to a token and contains its embedding vector. These vectors are typically dense, meaning that all the elements are non-zero, and they capture various semantic and syntactic properties of the token. When processing a token ID, the model performs a lookup operation in the embedding matrix to retrieve the corresponding embedding vector. For example, if the token ID 45 corresponds to “cat”, the embedding layer retrieves the 45th row in the matrix, which might look something like [0.25, −0.14, 0.72, . . . , 0.03] if the embedding dimension is 300. The output of the embedding layer of an LLM is a matrix of embeddings corresponding to all the tokens in the input sequence. If the input sentence is “The cat sits”, the output will be a matrix of shape (sequence_length, embedding_dim), where each row is the embedding vector for each token in the sequence.

At block 530, the embeddings are input into neural network layers of a an LLM (e.g., a first LLM). The neural network layers may include, for example, transformers, attention mechanisms, feed-forward networks, layer normalization, recurrent neural networks (RNNs), and so on. Each layer refines and transforms the input embeddings based on learned weights and input context. The neural network layers use these embeddings to perform tasks such as language modeling, text classification, and so on. After processing by each neural network layer, the output is still in the form of embeddings (e.g., dense vectors), which at this stage are enriched with contextual information. Accordingly, the embedding for a token now contains information not just about the token itself, but also about its relationship with other tokens in a sequence of tokens. The final output from the last neural network layer of the LLM is a set of contextualized embeddings. These embeddings are still in vector form and correspond to each token in the input sequence. However, they now represent the token in the context of the entire input sequence. These contextualized embeddings are typically passed through a linear layer (or a set of linear layers) to produce logits, which are raw scores for each token corresponding to the vocabulary size. The logits may then be processed by a softmax function to produce probabilities for each possible token in the vocabulary.

At block 535, the LLM generates an initial output in the form of tokens. In embodiments, the highest probability tokens from the softmax output of block 530 are often selected as a predicted output. These tokens represent the LLM's interpretation of the input sequence, typically as part of tasks like text generation, translation, or completion. The final output of the neural network layers in an LLM, before any post-processing like softmax, is a rich, context-aware vector representation for each token in the input sequence. These vectors are what the model uses to make its predictions.

At block 540, processing logic performs postprocessing of the output to format the output from tokens into a coherent text. After the neural network layers of a large language model (LLM) produce their final outputs (typically in the form of logits), several postprocessing steps are typically performed to generate the final output tokens or to interpret the model's predictions. In embodiments, processing logic performs token selection (decoding), which may include greedy decoding (where the model selects the token with the highest probability at each step), beam search (instead of choosing the highest probability token at each step, beam search keeps track of several sequences (beams) at once, exploring multiple potential paths and selecting the one with the overall highest probability), top-k sampling (model samples from the top k most probable tokens rather than always choosing the highest), top-p sampling (similar to top-k sampling, but instead of a fixed number of tokens, it samples from the smallest set of tokens whose cumulative probability exceeds a threshold p), temperature scaling, and/or other decoding schemes. Detokenization converts selected tokens (which may be in the form of IDs) back into human-readable text using the model's vocabulary. Additional postprocessing may also be performed, such as text postprocessing that includes adding/fixing punctuation and spacing, normalization, validation checks, and so on.

At block 545, processing logic may generate a final output of the LLM comprising search term(s) for the cognitive search results to treatment context understanding.

At block 550, processing logic provides a cognitive search request to a cognitive search service based on the final output of the LLM.

At block 555, processing logic receives treatment context information from the cognitive search service.

At block 560, processing logic generates a text prompt to the LLM or a second LLM based on the original prompt and the treatment context information. The prompt may optionally include a chat history and/or additional instructions based on the treatment context information.

At block 565, the LLM or the second LLM performs preprocessing of the text prompt.

At block 570, the LLM or the second LLM performs tokenization of the preprocessed text prompt.

At block 575, the LLM or the second LLM converts the tokens into embeddings.

At block 580, the LLM or the second LLM inputs the embeddings into neural network layers of the second LLM.

At block 585, the LLM or the second LLM generates an initial output in the form of embeddings.

At block 590, the LLM or the second LLM performs postprocessing of the output to format the output from tokens into coherent text.

At block 595, the LLM or the second LLM generates a final output comprising actionable recommendations.

At block 598, processing logic performs text to speech processing and outputs a speech response comprising the actionable recommendations. The speech response may be sent to a user device, which may output the speech response via speakers of the user device.

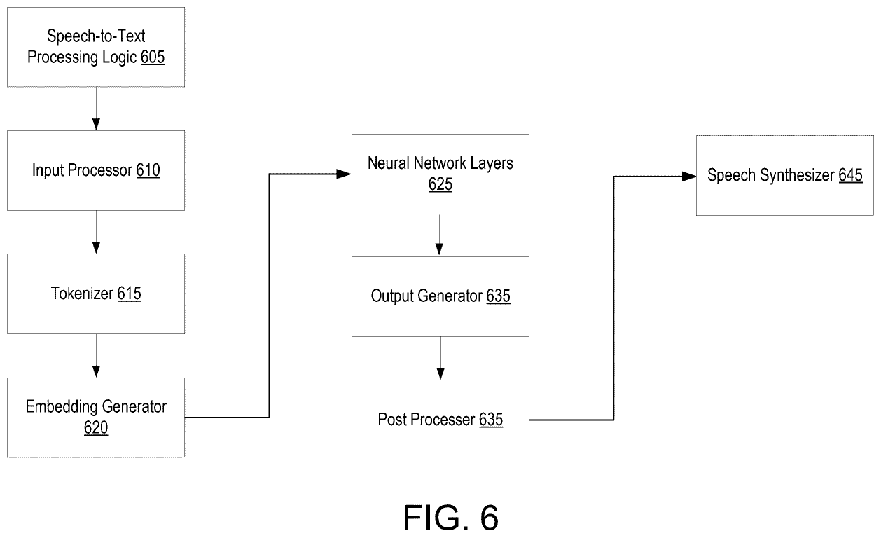

FIG. 6 illustrates a conceptual diagram of a large language model that, in combination with additional logic, may function as a medical virtual assistant, in accordance with an embodiment. The large language model includes one or more deep neural networks that may incorporate transformer architectures, which allow the LLM to process and generate sequences of text by attending to different parts of input and output sequences. The models includes multiple layers of neurons, each layer transforming the input data using weights and biases of the individual neurons that are learned during training. The LLM architecture may further include an attention mechanism that enables the LLM to weight the important of different words in a sentence relative to each other, allowing it to handle long-range dependencies in text.

In embodiments, the LLM includes a speech-to-text processing logic 605 (which may be considered a distinct component that is separate from the LLM). The speech-to-text processing logic may perform speech-to-text processing of input voice data. An input processor 610 may perform pre-processing of a text prompt, as previously discussed.

A tokenizer 615 may generate tokens from the preprocessed text, as discussed above. An embedding generator 620 may then generate embeddings for the generated tokens. The embeddings may be processed by the neural network layers 625. During training, an LLM processes vast amounts of text data and learns the statistical relationships between words, phrases, sentences, and even larger contexts. This is akin to recognizing patterns in the data-how words commonly co-occur, the structure of sentences, and the way ideas are typically expressed in natural language. An LLM predicts the next word or sequence of words based on the input it receives. This prediction is probabilistic, meaning the model calculates the likelihood of different possible continuations and selects the one with the highest probability. The model processes input as tokens (which may be words, subwords, or characters) and predicts the next token in the sequence. It does this by calculating the probability distribution over the possible tokens that could follow, given the preceding context.

In embodiments, the neural network layers include a transformer architecture with a self-attention mechanism that allows the model to weigh the importance of different parts of the input text when generating each word. For example, in a sentence like “The cat sat on the mat because it was tired,” the model can attend to “cat” when deciding what “it” refers to. The attention mechanism helps the model to focus on relevant parts of the input when making predictions, allowing it to handle long-range dependencies and complex sentence structures more effectively.

Artificial neural networks generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs). Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

In one embodiment, the neural network layers include a recurrent neural network (RNN). An RNN is a type of neural network that includes a memory to enable the neural network to capture temporal dependencies. An RNN is able to learn input-output mappings that depend on both a current input and past inputs.

An output generator 635 of the LLM may generate a final output of tokens based on outputs of the neural network layers 625. A post processor 635 of the LLM may then perform postprocessing of the output, as described above, producing a final text output that is human readable.

The LLM may include a speech synthesizer 645 that performs text-to-speech processing to transform the text output into a speech output. In embodiments, the speech synthesizer 645 may be considered a distinct component that is separate from the LLM.

Training of a neural network (e.g., neural network layers of an LLM) may be achieved in a supervised learning manner, which involves feeding a training dataset consisting of inputs (which may or may not be labeled) through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many inputs in the training dataset yields a network that can produce correct output when presented with inputs that are different than the ones present in the training dataset. In high-dimensional settings this generalization is achieved when a sufficiently large and diverse training dataset is made available. In some embodiments, neural network layers of an LLM are trained in an unsupervised learning manner.

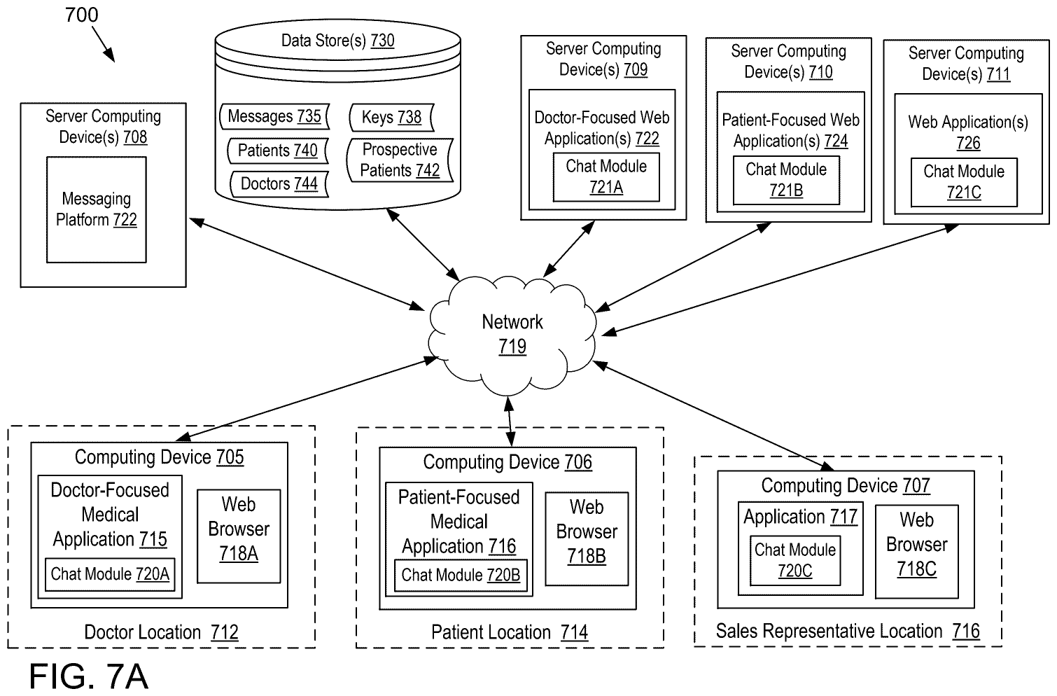

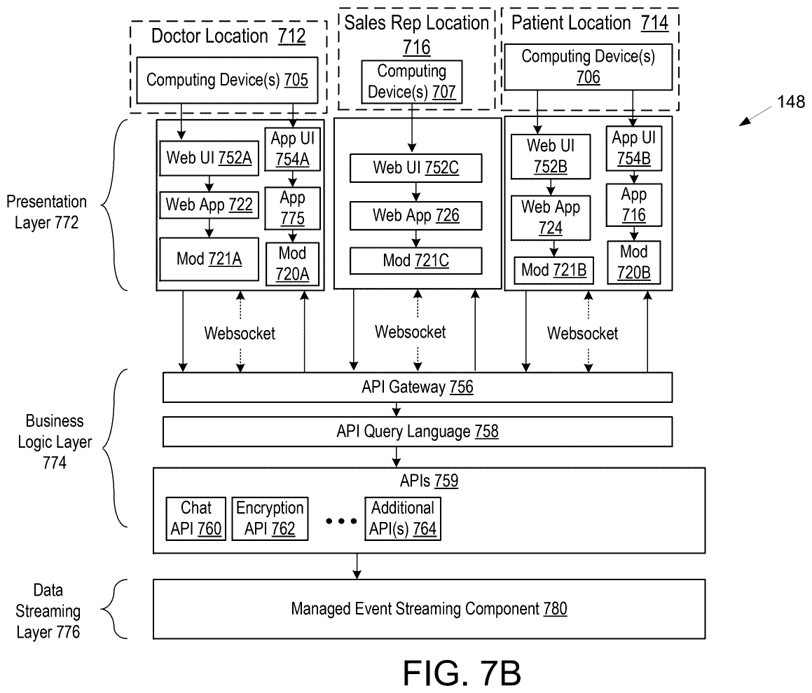

FIG. 7A illustrates a system 700 that enables communication between doctors and patients, in accordance with embodiments of the present disclosure. In embodiments, the system 700 includes one or more server computing devices 708 that execute a messaging platform 722. The messaging platform 722 may be an application-agnostic messaging platform that can enable communications (e.g., chats, messages, etc.) between multiple disparate applications, and in particular between different medical applications. Any application that has an appropriate chat module installed thereon can access the messaging platform 722 and communicate with other applications connected to the messaging platform.

Server computing device(s) 708 may each include one or more processing devices, memory, secondary storage, one or more input devices (e.g., such as a keyboard, mouse, tablet, and so on), one or more output devices (e.g., a display, a printer, etc.), and/or other hardware components. In embodiments, the server computing device(s) 708 include devices associated with a cloud computing service. Cloud computing services provide on-demand access to computing resources over the internet, including servers, storage, databases, networking, software, analytics, and intelligence. Cloud computing uses virtualization technology to divide physical hardware into multiple virtual machines (VMs). Each VM can run its own operating system and applications independently, allowing multiple users or organizations to share the same physical resources. The cloud computing service may include an infrastructure as a service (IaaS) that provides virtualized computing resources, a platform as a service (PaaS) that offers a platform on which applications can be developed, run and managed, and/or software as a service (SaaS) that delivers software applications over a network.

In embodiments, the server computing devices 708 are connected either directly or via a network 719 to one or more data stores 730. The server computing devices 708 may additionally be connected to other server computing devices 709, 710 and/or other computing devices 705, 706, 707 via network 708. The network 719 may be a local area network (LAN), a public wide area network (WAN) (e.g., the Internet), a private WAN (e.g., an intranet), a wireless network of a wireless carriers (e.g., a 3G, 4G or 5G wireless network), or a combination thereof.

Data store(s) 730 may include one or more databases (e.g., structured databases), file systems, and/or other storage mechanisms for storing data used by and/or associated with messaging platform 722. In embodiments, data store(s) 730 store messages 735 between parties (e.g., between a doctor and a patient), information on doctors 744 having accounts with the messaging platform 722 and/or with one or more medical applications, information on patients 740 having accounts with the messaging platform 722 and/or with one or more medical applications, information connecting particular patients to particular doctors (e.g., indicating, for each doctor, a list of patients of that doctor), security keys 738 used for messaging, information on prospective patients 742 having accounts with the messaging platform 722 and/or with one or more medical applications, information connecting particular prospective patients 742 to particular doctors 744, and so on. In embodiments, the messages 735 include message threads, and may include saved prior messages between two parties (e.g., between a doctor and a patient). A different message thread (also referred to as a channel) may be created for each unique pair of individuals. Each message thread may contain a history of messages exchanged between the two parties, including optionally any files (e.g., images, videos, etc.) shared between the parties.

Each of computing device 705, computing device 706, computing device 707, server computing device(s) 709 and/or server computing device(s) 710 may use messaging platform 722 to communicate with one another and/or to provide messaging between users of one or more applications that have chat modules for interfacing with messaging platform 722 installed thereon. Each of computing devices 705, 706, 707 may be a mobile computing device (e.g., such as a laptop computer, a tablet computer, a mobile phone, etc.) or a traditionally stationary computing device (e.g., such as a desktop computer, a smart television, a computing device of an intraoral scanning system that pairs with an intraoral scanner), and so on. Each computing device 705, 706, 707 and/or server computing device(s) 709, 710 may include one or more processing devices, memory, secondary storage, one or more input devices (e.g., such as a keyboard, mouse, tablet, and so on), one or more output devices (e.g., a display, a printer, etc.), and/or other hardware components.

Computing device 705 may be a computing device of a doctor, may be at a doctor location 712, and may include a doctor-focused medical application 715 installed thereon. The doctor-focused medical application 715 may include one or more functions of interest to a doctor, such as functionality to schedule an appointment with a patient, to view a patient's current treatment status, to perform intraoral scanning of a patient, to generate a treatment plan for a patient, to view a medical history of a patient, and so on.

In one embodiment, the doctor-focused medical application 715 is an orthodontic treatment planning application. One example of an orthodontic treatment planning application that may be used is Align Technology's ClinCheck® Pro software. The Orthodontic treatment planning application may enable doctors to review, modify and approve treatment plans. For example, the orthodontic treatment planning application may enable a doctor to submit patient case details (e.g., 3D models of patient dental arches), may process the patient case details to generate a treatment plan, may forward the patient case details to a lab or other facility to enable the lab or other facility to generate a treatment plan, may display a generated treatment plan (e.g., including one or more treatment stages, each including distinct 3D models of the patient's teeth at a treatment stage), may provide tools for the doctor to change the treatment plan (e.g., to change one or more stages of the treatment plan), and so on. Via the orthodontic treatment planning application, the doctor may make treatment plan modifications themselves, view side-by-side comparisons, and customize treatment plans for specific cases. The doctor can directly edit, undo, redo, and investigate new approaches to treatment plans quickly, with or without involving a technician. The orthodontic treatment planning application may provide 3D controls that enable precise modifications to treatment plans directly on a patient's dentition in real time. The 3D controls may include controls for multi-tooth adjustments, arch-form, attachments and cuts, interproximal reduction and spacing. These tools provide precise control over final tooth position to help doctors quickly achieve treatment goals. In some embodiments, the orthodontic treatment planning application provides CBCT integration, and may auto-generate 3D models of dental arches with roots, crowns, and bone for more-informed treatment planning. In some embodiments, the orthodontic treatment planning application realistically simulates what a patient's post-treatment face will look like, allowing doctors to communicate treatment plans to their patients more effectively, and enabling patients to visualize their treatment outcomes. For example, the orthodontic treatment planning application may receive a brief (e.g., 15-30 second) video of a patient smiling and/or talking, and may generate a modified version of the video in which the patient is smiling and/or talking with their post-treatment dentition.

In one embodiment, the doctor-focused medical application 715 is dental practice application, which may interface with a doctor portal for dental and/or orthodontic treatment. The doctor portal may be, for example, a doctor focused web application 722 in embodiments. For example, doctor-focused web application 722 may provide dentists and orthodontists access to tools for managing their practice and patient care. Through the portal, doctors can view treatment plans, access case files, upload patient images, and monitor patient progress. The dental practice application may help streamline tasks like patient photo uploads, virtual care, and patient consultations. This allows doctors to manage patient cases, track treatment progress, and engage with potential and existing patients remotely, improving overall workflow and patient experience.

In one embodiment, the doctor-focused medical application 715 is an intraoral scanning application. In such an example, computing device 705 may be connected to and/or paired with an intraoral scanner (not shown). The intraoral scanner may include a probe (e.g., a hand held probe) for optically capturing three dimensional structures of a patient's dentition. One example of such an intraoral scanner is the iTero® intraoral digital scanner manufactured by Align Technology, Inc. The intraoral scanner may be used to perform an intraoral scan of a patient's oral cavity. Doctor-focused medical application 715 may be an intraoral scan application running on computing device 705, which may communicate with the intraoral scanner to effectuate the intraoral scanning. A result of the intraoral scanning may be a sequence of intraoral images or scans that have been generated. Each intraoral scan may include x, y and z position information for one or more points on a surface of a scanned object (e.g., of a patient's upper and lower jaw). The intraoral scanner may transmit the intraoral scans to the computing device 505. In addition to 3D surface data (e.g., in the form of intraoral scans that may be one or more point clouds), the intraoral scanner may additionally capture and transmit to the computing device 2D or 3D color image data, near infrared (NIR) image data, ultraviolet image data, and so on. In one embodiment, the intraoral scan application generates a virtual 3D model of the scanned dental site. To generate the virtual model, the intraoral scan application may register and “stitch” together the intraoral scans generated from the intraoral scanning session. In one embodiment, performing registration includes capturing 3D data of various points of a surface in multiple scans (views from a camera), and registering the scans by computing transformations between the images. The generated 3D model(s) of the patient's dental arch(es) may be provided to a treatment planning application for generation of an orthodontic treatment plan in embodiments, as described above.

In one embodiment, doctor focused medical application 715 may be a practice management system. The practice management system, such as for a dental office, may manage patient records, patient billing, appointment scheduling, insurance claims, and so on. For example, a practice management system may manage electronic health records, such as clinical notes (e.g., documentation of each patient visit, including what procedures were performed, observations, and recommendations), treatment progress (e.g., tracking of ongoing treatments with timelines, milestones, and outcomes), digital imaging (e.g., storage and access to digital x-rays, 3D scans, intraoral photos, and other diagnostic images), prescriptions (e.g., including refill tracking), and/or referrals (e.g., documentation and tracking of referrals to specialists or other healthcare providers). The practice management system may provide inventory and supply management, such as inventory tracking (e.g., monitoring of dental supplies, orthodontic appliances and prosthodontic materials), automated reordering (e.g., triggering reorders when inventory levels fall below a certain threshold), and/or supplier management (e.g., tracking orders, deliveries and supplier performance).

In a non-limiting example, a patient who wishes to straighten their teeth may opt for Invisalign® treatment. Invisalign is a process that creates a custom made series of clear aligners specifically for the patient. The clear aligners are worn over the patient's teeth and gradually shift the patient's teeth. A new set of aligners may be worn after a specified period of time (e.g., two weeks) until treatment is complete.

The patient may visit a dental practitioner or orthodontist to begin Invisalign treatment. The dental practitioner may utilize a scanning system including an intraoral scanner and computing device 705 to scan the patient's teeth in a scanning mode. The dental practitioner may use the scanner to capture the patient's teeth segments (e.g., upper arch, lower arch, bite segments) in one or more sets of intraoral scans. The medical application 715 may register and stitch together the intraoral scans to create a 3D rendering of the scanned segments and present the 3D rendering to the dental practitioner on the user interface of the medical application. Once the scans are completed, the dental practitioner may next navigate to an image processing mode, which may generate a virtual 3D model by registering and stitching together the intraoral images. Once an adequate set of 3D renderings and/or virtual 3D model are complete, the 3D renderings and/or 3D models may be saved to a patient profile.

The dental practitioner may then provide input to switch to a planning mode and/or to launch another doctor-focused medical application to perform treatment planning, in which a final tooth arrangement may be determined and one or more intermediate tooth arrangements may be determined. A treatment plan may be generated to provide a progression of treatment stages from the patient's initial tooth arrangement to the target final tooth arrangement, where a separate 3D model is associated with each treatment stage.

Once an adequate set of 3D models is generated, the 3D models may be saved to a patient profile. The dental practitioner may then navigate to a delivery mode to electronically send the completed patient profile to a processing center. The processing center may then generate the custom made series of clear aligners for the patient and deliver the clear aligners to the dental practitioner. The patient would then return to the dental practitioner to receive the first set of clear aligners and verify the clear aligners properly fit onto the patient's teeth.

The doctor-focused medical application 715 may include an integrated chat module 720A. The chat module 720A provides functionality for accessing and using messaging platform 722 in embodiments. The chat module 720A may provide a separate display or window associated with messaging in embodiments, which may not be available on the doctor-focused medical application 715 without the chat module 720A. The separate display or window may include, for example, functionality and associated icons, text boxes, drop down menus, etc. that enable a doctor to see a list of patients that they can communicate with, initiate a chat with a selected patient, send a message to a selected patient, receive and view messages from the selected patient, read/view a message history between the doctor and the selected patient, and so on.

The chat module 720A may have been compiled from a chat service developer's kit (SDK). The SDK may include all of the necessary information to compile chat module 720A and enable chat module 720A to both integrate with doctor-focused medical application 715 and to connect to messaging platform 722. For example, messaging platform 722 may include one or more application programming interfaces (APIs), and chat module 720A may include code for accessing and interfacing with the one or more APIs of the messaging platform 722. In some embodiments, the SDK used to compile chat module 720A is a platform-agnostic SDK. Accordingly, the same SDK may be used to compile a chat module for use on devices running the Android operating system, the iOS operating system, the Windows operating system, the Linux operating system, and so on. In some embodiments, the SDK can be used to compile chat modules for each of the iOS operating system and the Android operating system.

In some embodiments, computing device 705 may not include a local doctor-focused medical application 715 installed thereon, or a doctor may choose not to use the doctor-focused medical application 715. For such instances, computing device 705 may include a web browser 718A (e.g., such as Mozilla Firefox, Google Chrome, Apple Safari, etc.) installed thereon. The web browser 718A may be used to access one or more doctor-focused web application(s) 722. The doctor-focused web applications 722 may provide any of the functionality earlier described with reference to doctor-focused medical application 715 in embodiments. For example, doctor-focused web application(s) 722 may include a treatment planning application, an intraoral scanning application, and so on. The doctor-focused web application(s) 722 may also include a chat module 721A that enables interfacing with messaging platform 722. The chat module 721A may provide similar functionality to chat module 720A, but in some embodiments may be compiled from a different SDK from chat module 720A. For example, chat module 720A may be compiled from an SDK for mobile devices, and chat module 721A may be compiled from an SDK for web applications, such as a Javascript SDK. In one embodiment, chat module 721A is a Javascript web widget.

In some embodiments, computing device 705 is configured to understand one or more voice commands for controlling one or more functions of doctor-focused medical application, such as use of the above described functions of any of the above described doctor-focused medical applications. The voice commands may be provided to computing device 705 via a microphone of the computing device 705 in some embodiments. For example, if computing device 705 is a mobile phone, then a microphone of the mobile phone may be used to capture a voice command. In some embodiments, the voice command and/or other speech input (e.g., a message to be transcribed) is input via a wearable IoT device or AR display. In one example in which computing device 705 is part of an intraoral scanning system that includes an intraoral scanner, the intraoral scanner may include a microphone, and the user may input voice commands to the computing device for controlling functions of the doctor-focused medical application 715 via voice commands captured using the microphone of the intraoral scanner.

In some embodiments, the doctor-focused medical application 715 includes multiple voice-activated functions. The voice-activated functions may be registered with the computing device 705 such that the voice-activated functions can be initiated even when the doctor-focused medical application 715 is not running. For example, an operating system running on computing device 705 may include an application intents framework that provides a programmatic way to make an application's content and functionality available to system services of the operating system. The application intents framework may enable applications such as doctor-focused medical application 715 to expose its capabilities, metadata, user interface information, activation phrases, and/or other information usable to initiate actions and/or functions of the application (e.g., of doctor-focused medical application 715). In embodiments, multiple different functions of doctor-focused medical application 715 are registered with the application intents framework of the operating system running on computing device 705. For each function of doctor-focused medical application 715, one or more words and/or phrases are associated with the function. Each function may have its own set of words and/or phrases associated with it. In embodiments, a patient does not need to speak the words or phrases associated with a function exactly to initiate the function. The doctor may use alternative language that conveys the same meaning and/or that is similar to the registered phrases/words, which may trigger the associated function.

Once one or more functions of doctor-focused medical application 715 are registered with the application intents framework, at any time (whether or not the doctor-focused medical application 715 is running), the computing device 705 may receive a voice instruction associated with a function of the doctor-focused medical application 715 via a microphone of the computing device 705. The computing device 705 may process the voice instruction to determine that the voice instruction is for the function of the medical application. The computing device 705 may then cause the medical application 715 to perform the function, and may generate an output responsive to causing the medical application to perform the function.

In one embodiment, the voice-activated function is a messaging function provided by chat module 720A. The doctor may provide a voice instruction to activate a message function, and may dictate a message verbally in some embodiments. The doctor may verbally instruct the message to be sent to a particular patient, which may trigger chat module 720A to interface with messaging platform 722 to send the message.

In embodiments, the doctor-focused medical application 715 may use one or more speech to text transcription functions or services (e.g., which may execute on server computing device(s) 709) to transcribe the message. In one embodiment, the doctor-focused medical application 715 may then use text to speech services to regenerate the original audio message and may play that audio message back to the patient to ensure that the message is accurate and give the patient a chance to alter the message in some embodiments. Once the doctor is satisfied with the message, they may provide a verbal instruction to proceed with sending the message. In response, the doctor-focused medical application 715 may use chat module 720A to send the message to the patient focused medical application 716 via messaging platform 722 in embodiments.

Computing device 706 may be a computing device of a patient or prospective patient, may be at a patient location 714, and may include a patient-focused medical application 716 installed thereon. The patient-focused medical application 716 may include one or more functions of interest to a patient, such as functionality to schedule an appointment with a doctor, to view their current treatment status, to find a doctor, and so on.

In one embodiment, the patient-focused medical application 715 is virtual care application. The virtual care application may track a patient's treatment, may provide insurance verification for treatments, may provide information to enable a prospective patient to find a doctor, may show a prospective patient or patient what they might expect post treatment, may provide educational information on treatments, and so on. In one embodiment, the patient-focused medical application 715 is an orthodontic treatment virtual care application, an example of which is the My Invisalign™ app provided by Align Technology, Inc. The orthodontic virtual care application may have multiple functions of interest to a patient or prospective patient. For example, the orthodontic virtual care application may include a function that enables an individual to take a photo or video of their smile. The application may then process the photo or video (or send the photo or video to a server for processing) to generate a synthetic image of their smile after orthodontic treatment is performed, which may be shown via the application. The orthodontic virtual care application may include a “find a doctor” feature that may show doctors that are near the individual who are capable of performing one or more types of treatments of interest to the patient. The orthodontic virtual care application may include a “check your insurance” feature that enables the individual to check whether their insurance will cover orthodontic treatment and/or what portion of orthodontic treatment their insurance will cover. The orthodontic virtual care application may provide educational information on how a polymeric orthodontic aligner system works, how much it costs, how it differs from traditional braces, how long treatment takes, and so on.

In some embodiments, the patient-focused medical application 715 is an orthodontic virtual care application that comprises multiple functions associated with orthodontic treatment. In one embodiment, the functions include a timer that times an amount of time that an orthodontic aligner has been removed from a mouth of the patient. The timer may include one or more preset time periods and/or may be manually set by the patient. When the timer is activated, it may count down, and the medical application may determine when the timer has elapsed and may output an alarm (e.g., via a speaker of computing device 706) responsive to determining that the timer has elapsed. In one embodiment, the functions include an orthodontic aligner tracker that tracks a stage of orthodontic treatment. The orthodontic aligner tracker may output (e.g., to a display and/or via audio) an identification of a current orthodontic aligner being used by the patient. In one embodiment, the functions include a doctor finder. The doctor finder may determine a current location of a patient, and determine one or more nearby doctors that can provide orthodontic treatment for the patient. Information on the nearby doctors, including their names, contact information, locations (e.g., on a map), and so on may be output by patient-focused medical application 715 (e.g., via a display and/or audio). Other functions that may be performed by patient-focused medical application 716 include requesting and/or setting an appointment with a doctor, creating an event for an appointment in a calendar application (e.g., that may run on computing device 706), accepting instructions sent by a doctor, changing a current orthodontic aligner being worn by a patient to a different orthodontic aligner, changing a total number of orthodontic aligners to be worn by the patient for orthodontic treatment, extending a date at which the patient is to transition from a current orthodontic aligner to a next orthodontic aligner (e.g., by 1 day, by 2 days, by 5 days, by 1 week, etc.), sending a message to a doctor, sharing photos with the doctor, ordering a retainer or canceling a subscription for a retainer, and so on.

In one embodiment, the functions include a patient smile image capture function. The patient smile image capture function may include a user interface that is output to a display of the computing device 706. An image capture device of the computing device 706 may capture an image of a dentition of the patient, and may transmit the image of the dentition of the patient to computing device 705 associated with a doctor of the patient. In some embodiments, the patient smile image capture function may perform a comparison of the image of the current smile of the patient to a prior image of a past smile of the patient, or send the image to server computing device 710 to enable the server computing device 710 to perform the comparison. Patient-focused medical application 716 may generate and/or receive a comparison result, and may output the comparison result via a display of computing device 706. The comparison result may include one or more observations, such as observations indicating whether treatment is progressing as planned, whether treatment is progressing slower than planned, whether treatment is progressing faster than planned, whether any problems have been identified, and so on. In some embodiments, patient-focused medical application 716 or patient-focused web application 724 performs segmentation of the current and/or past images of the patient's smile. The segmentation information may be displayed as an overlay on the current and/or past images, and may identify individual teeth, changes to individual teeth, gingiva, and so on.

In some embodiments, the patient-focused medical application 716 includes multiple voice-activated functions. The voice-activated functions may be registered with the computing device 706 such that the voice-activated functions can be initiated even when the patient-focused medical application 716 is not running. For example, an operating system running on computing device 706 may include an application intents framework that provides a programmatic way to make an application's content and functionality available to system services of the operating system. The application intents framework may enable applications such as patient-focused medical application 716 to expose its capabilities, metadata, user interface information, activation phrases, and/or other information usable to initiate actions and/or functions of the application (e.g., of patient-focused medical application 716). In embodiments, multiple different functions of patient-focused medical application 716 are registered with the application intents framework of the operating system running on computing device 706. For each function of patient-focused medical application 716, one or more words and/or phrases are associated with the function. Each function may have its own set of words and/or phrases associated with it. In embodiments, a patient does not need to speak the words or phrases associated with a function exactly to initiate the function. The patient may use alternative language that conveys the same meaning and/or that is similar to the registered phrases/words, which may trigger the associated function.

Once one or more functions of patient-focused medical application 716 are registered with the application intents framework, at any time (whether or not the patient-focused medical application 716 is running), the computing device 706 may receive a voice instruction associated with a function of the patient-focused medical application 716 via a microphone of the computing device 706. The computing device 706 may process the voice instruction to determine that the voice instruction is for the function of the medical application. The computing device 706 may then cause the medical application 716 to perform the function, and may generate an output responsive to causing the medical application to perform the function.

In one example, the voice instruction comprises a request for identification of a current aligner being used by the patient. In response, an orthodontic aligner tracker function of the patient-focused medical application 716 may determine the current aligner being used by the patient, and the patient-focused medical application 716 may generate an output that comprises the identification of a current orthodontic aligner being used by the patient.

In one example, the voice instructions comprises a request for information on when a current orthodontic aligner being used by the patient is to be replaced by a next orthodontic aligner. In response, an orthodontic aligner tracker of the patient-focused medical application 716 that tracks a stage of orthodontic treatment may determine when the current orthodontic aligner being used by the patient is to be replaced by the next orthodontic aligner. The patient-focused medical application 716 may then output an indication or notice of when the current orthodontic aligner is to be replaced.

In one example, the voice instruction is an instruction to set a timer, to start the timer, or to stop the timer. The patient-focused medical application 716 may perform the requested function with respect to the timer, such as setting the timer, starting the timer, or stopping the timer. If the timer expires (e.g., counts down to zero), then the patient-focused medical application 716 may output an alarm, which may indicate to the patient that they need to insert the orthodontic aligner into their mouth.

In one example, the voice instruction is an instruction to find a doctor for the patient. For example, the voice instruction may comprise a request for information on nearby doctors that can provide orthodontic treatment for the patient. A doctor finder function of the patient-focused medical application 716 may determine one or more nearby doctors that can provide orthodontic treatment for the patient, and the patient-focused medical application 716 may output information on the nearby doctors.

In one example, the voice instruction is an instruction to launch a patient smile image capture function of the patient-focused medical application 716. In response to such a verbal instruction, computing device 706 may launch the patient smile image capture function. The patient smile image capture function may then generate one or more images of the patient's smile, provide instructions on how to position a camera of the computing device 706, how the patient should pose, and so on. Once one or more images are captured, they may be transferred to a computing device of a doctor (e.g., optionally using messaging platform 722), may be processed to generate results that may be output, and so on.

In one example, the voice instruction is an instruction to schedule an appointment with a doctor. Such a voice instruction may cause an appointment schedule function of the patient-focused medical application 716 to be invoked. The appointment schedule function may interface with computing device 705 of the doctor to schedule an appointment with the doctor. If an appointment is successfully scheduled, then the patient-focused medical application 716 may output a verbal confirmation of the appointment, optionally including the appointment date and time. If an appointment is not successfully scheduled, the patient-focused medical application 716 may output a notice that a schedule could not be scheduled. In some instances, the patient-focused medical application 716 may output a question of whether the patient would like to call the doctor's office to schedule the appointment. If the patient answers in the affirmative, then the patient-focused medical application 716 may determine a phone number of the doctor's office and automatically call the doctor's office using the determined phone number. If an appointment is successfully scheduled, patient-focused medical application 716 may automatically create an event for the appointment in a calendar application or service.

In one embodiment, the voice instruction is an instruction to send a message to the doctor. Responsive to such an instruction, the patient may dictate their message to the doctor, which may be recorded by the patient-focused medical application 716. The patient-focused medical application 716 may use one or more speech to text transcription functions or services (e.g., which may execute on server computing device(s) 710) to transcribe the message. In some embodiments, the patient-focused medical application 716 may then use text to speech services to regenerate the original audio message and may play that audio message back to the patient to ensure that the message is accurate and give the patient a chance to alter the message. Once the patient is satisfied with the message, they may provide a verbal instruction to proceed with sending the message. In response, the patient-focused medical application 716 may use chat module 720B to send the message to the doctor focused medical application 715 via messaging platform 722 in embodiments.

Some examples of virtual care medical applications are described in U.S. Pat. No. 10,248,883, issued Apr. 2, 2019, entitled “Photograph-based Assessment of Dental Treatments and Procedures,” which is incorporated by reference herein. Some examples of virtual care medical applications are described in U.S. Pat. No. 11,800,216, issued Oct. 24, 2023, entitled “Image-based orthodontic treatment refinement,” which is incorporated by reference herein. Some examples of virtual care medical applications are described in U.S. Pat. No. 11,589,957, issued Feb. 28, 2023, entitled “Methods and Apparatuses for Dental Images,” which is incorporated by reference herein. The functionality of any of the systems described in each of these referenced patents may be associated with a voice command that may be used to trigger such functionality in embodiments.

In some instances, if the patient-focused medical application 716 is not already running on computing device 706, responsive to receiving a verbal instruction to perform a function of the patient-focused medical application that patient-focused medical application 716 is launched and the requested function is performed. In some embodiments, patient-focused medical application 716 may be protected by security measures that may us a password, a personal identification number (PIN) or biometric information to secure the patient-focused medical application 716. Responsive to identifying a function of patient-focused medical application 716 to be executed, computing device 706 may determine that patient-focused medical application 716 is protected, and may output an audio prompt (and/or visual prompt) for a personal identification number (PIN) or a password to be entered. For example, an audio prompt may be output via the speaker. The patient may provide a verbal input in which the patient recites a sequence of characters (e.g., letters and/or numbers) that include the PIN or password for the patient-focused medical application 716 via a microphone of computing device 706. The computing device 706 may determine whether the sequence of characters matches the PIN or the password. If the sequence of characters does not match the PIN or password, then the patient-focused medical application 716 may not be launched, and the requested function may not be performed. In some embodiments, an error is output and the patient is asked to reinput the PIN or password. This may be repeated a number of times until the patient-focused medical application 716 is locked out for some period of time or a correct PIN or password is provided. Once the provided sequence of characters is determined to match the PIN or password, computing device 706 may load the patient-focused medical application 716 and cause the patient-focused medical application 716 to perform the requested function.

In some instances, one or more widgets associated with functionality of patient-focused medical application 716 are installed on computing device 706. Widgets may be small, interactive applications that can perform limited functions and/or display useful information. Unlike full applications that generally need to be opened to access their features, widgets execute simple actions without requiring opening of an application. In some embodiments, responsive to receiving a voice command associated with a function of patient-focused medical application 716, computing device 706 determines whether a widget associated with the function is installed on computing device 706. If such a widget is installed on computing device 706, then rather than launching the patient-focused medical application 716, computing device may identify the widget associated the function and invoke the widget. The widget may then cause the medical application to perform the function or may perform the function for the medical application.

In some embodiments, a widget associated with a function of patient-focused medical application 716 may be installed on computing device 706. In such embodiments, the widget may perform the requested function without loading the patient-focused medical application 716, which may bypass a need for the patient to input their PIN or password. For example, functions provided by widgets associated with patient-focused medical application 716 may not include sensitive information of the patient, and may be invoked without requiring the patient to input a PIN or password. Accordingly, in embodiments widgets may streamline voice commands of certain functions for patient-focused medical application 716.

The patient-focused medical application 716 may include an integrated chat module 720B. The chat module 720B provides functionality for accessing and using messaging platform 722 in embodiments. The chat module 720B may provide a separate display or window associated with messaging in embodiments, which may not be available on the patient-focused medical application 716 without the chat module 720B. The separate display or window may include, for example, functionality and associated icons, text boxes, drop down menus, etc. that enable a patient to see a list of doctors in their area, initiate a chat with a selected doctor, send a message to a doctor, receive and view messages from the doctor, read/view a message history between the doctor and the patient, and so on. In some embodiments, computing device 706 includes a widget for chat module 720B. Accordingly, one or more functions of chat module 720B may be invoked verbally without loading patient-focused medical application 716 in some embodiments.

Like chat module 720A, chat module 720B may have been compiled from a chat service developer's kit (SDK). For example, chat module 720A and chat module 720B may have been compiled from a same SDK in some embodiments.

In some embodiments, computing device 706 may not include a local patient-focused medical application 716 installed thereon, or a patient or prospective patient may choose not to use the patient-focused medical application 716. For such instances, computing device 706 may include a web browser 718B (e.g., such as Mozilla Firefox, Google Chrome, Apple Safari, etc.) installed thereon. The web browser 718B may be used to access one or more patient-focused web application(s) 724. The patient-focused web applications 724 may provide any of the functionality earlier described with reference to patient-focused medical application 716 in embodiments. For example, patient-focused web application(s) 724 may include a virtual treatment application. The patient-focused web application(s) 724 may also include a chat module 721B that enables interfacing with messaging platform 722. The chat module 721B may provide similar functionality to chat module 720B, but in some embodiments may be compiled from a different SDK from chat module 720B. For example, chat module 720B may be compiled from an SDK for mobile devices, and chat module 721B may be compiled from an SDK for web applications, such as a Javascript SDK.

Computing device 707 may be a computing device of a sales representative, business representative, or service representative of a medical device company or medical services company, may be at a sales representative location 716, and may include an application 717 installed thereon. The application 717 may include one or more functions of interest to a sales representative, such as functionality to complete orders for doctors and/or patients, to check order status (e.g., for orthodontic aligners, for retainers, for intraoral scanners, for protective sleeves of intraoral scanners, and so on), for guiding doctors through use of complex medical applications, and so on.

The application 717 may include an integrated chat module 720C. The chat module 720C provides functionality for accessing and using messaging platform 722 in embodiments. The chat module 720C may provide a separate display or window associated with messaging in embodiments, which may not be available on the application 717 without the chat module 720C. The separate display or window may include, for example, functionality and associated icons, text boxes, drop down menus, etc. that enable a sales representative to see a list of doctors in their service area, initiate a chat with a selected doctor, send a message to a doctor, receive and view messages from the doctor, read/view a message history between the doctor and the sales representative, and so on.

Like chat module 720A and 720B, chat module 720C may have been compiled from a chat service developer's kit (SDK). For example, chat module 720A, chat module 720B and chat module 720C may have been compiled from a same SDK in some embodiments.

In some embodiments, computing device 707 may not include a local application 717 installed thereon, or a sales representative may choose not to use the application 717. For such instances, computing device 707 may include a web browser 718C (e.g., such as Mozilla Firefox, Google Chrome, Apple Safari, etc.) installed thereon. The web browser 718C may be used to access one or more web application(s) 726 that may provide particular functionality for a medical device or services representative. The web applications 726 may provide any of the functionality earlier described with reference to application 717 in embodiments. The web application(s) 726 may also include a chat module 721C that enables interfacing with messaging platform 722. The chat module 721C may provide similar functionality to chat module 720C, but in some embodiments may be compiled from a different SDK from chat module 720C. For example, chat module 720C may be compiled from an SDK for mobile devices, and chat module 721C may be compiled from an SDK for web applications, such as a Javascript SDK.

Messaging platform 722 provides instant messaging functionality to disparate applications that include an appropriate chat module installed thereon for interfacing with messaging platform 722, including doctor-focused medical application 715, patient-focused medical application 716, application 717, doctor-focused web application 722, patient-focused web application 724 and web application 726. Messaging platform 722 may include one or more APIs associated with chat functionality, security, notifications, events, user profiles, doctors, and so on. Each of the APIs may be responsible for different functionality of the messaging platform 722. In some embodiments, messaging platform 722 includes an API gateway (e.g., such as the WSO2 API gateway) that manages, secures and scales API calls. The API gateway may receive all API requests from any of the chat modules 720A-C, 721A-C in embodiments, and may forward the API requests to appropriate APIs. The messaging platform 722 may interface with data store 730 for storage of messages 735, patient information 740, doctor information 744, security keys 738 and/or prospective patient information 742 in embodiments.

Messaging platform 722 facilitates the communication between a consumer (e.g., patient or prospective patient) and a doctor (or staff of the doctor) related to treatment options and/or services. This helps the doctor and staff to provide improved customer service. Messaging platform 722 also helps facilitate conversation between a sales representative (e.g., territory manager) and a doctor or staff for any new or ongoing business updates.

In some embodiments, the messaging platform 722 enables transmission and storage of images (e.g., pictures of a patient's teeth, smile, dental arches, etc.), videos (e.g., videos of the patient's teeth, smile, dental arches, etc.), and/or other files. Such files may be appended to messages in embodiments, and the files may be included as part of messages that are exchanged via messaging platform 722. In some embodiments, messaging platform 722 generates a preview of the content of uploaded files, and includes the preview in a message.

In some embodiments, files such as images, assessment results of images, etc. are uploaded, stored, analyzed, etc. outside of the context of the messaging platform 722. In such embodiments, medical applications (e.g., doctor-focused web applications 722) may receive such data outside of messaging platform 722, and messaging platform 722 may be informed of the data. Messaging platform 722 may then append links to the data, previews of the data, etc. in messages in some instances. Additionally, in some instances an application may automatically generate a message responsive to a file such as an image of a patient being uploaded to the application, and the message may be sent to a doctor and/or patient via messaging platform 722.

In an example, a user or doctor may upload an image of a patient's smile or a doctor may upload a 3D model of the patient's dental arch(es) to a doctor focused web-application 722 or patient-focused web application 724 without using chat module 721A. The doctor-focused web application 722 or patient-focused web application 724 may use one or more trained machine learning models to segment the input image and/or 3D model into objects such as a face, teeth, gingiva, and/or other dental objects or conditions. In some instances, a patient may be actively taking a video or one or more pictures of their face, and one or more machine learning models may evaluate captured images/video and output suggestions for the patient that will improve a quality of captured images (e.g., to smile wider, rotate head, move to better lit area, etc.).

In embodiments, when files (e.g., images) are uploaded to a doctor-focused web application 722 or patient-focused web application 724 (e.g., optionally via a patient-focused medical application 716 or doctor-focused medical application 715), a notification about the uploaded files/images a notification may be sent to the doctor and/or patient about the files/images via the messaging platform 722. For example, a message may be automatically generated and sent notifying the patient/doctor that the images have been uploaded.

In some cases, a sales representative may provide credentials to log into application 717 and/or web application 726. By logging into the application 717 or web application 726, a token may be generated, which may be used to access chat module as well as a database or other data store that provides regional territory information (e.g., such as doctors in a region that the sales representative is responsible for). Accordingly, a shared token may be used both for accessing the data store and for using the messaging platform 722 in embodiments.

The messaging platform and associated systems are described in greater detail with reference to FIG. 7B.

FIG. 7B illustrates an application architecture 748 for a messaging platform, in accordance with an embodiment. The messaging platform may correspond to messaging platform 722 of FIG. 7A in embodiments.

The architecture 748 for the messaging platform may include a presentation layer 772, a business logic layer 774 and a data streaming layer 776. The presentation layer 772 is responsible for displaying data, handling user input, and communication with business logic layer 774. The presentation layer 772 may be provided by one or more front-end applications, which may run on local computing devices 705, 706, 707 of one or more end users of the messaging platform and/or on one or more server computing devices 709, 710, 711. In embodiments, different types of users may access the messaging platform via different applications and/or devices.

In an example, a doctor at doctor location 712 may include one or more computing devices 705, which may include a desktop computer, a laptop computer, a mobile phone, a tablet computer, and so on. In some embodiments, the computing device 705 uses a locally running web browser to access a web user interface (UI) 752A of doctor-focused web application 722. Alternatively, or additionally, the computing device 705 may include doctor-focused medical application 715 installed thereon, and may access the doctor-focused medical application 715 via an application UI 754A of the doctor-focused medical application 715. The doctor-focused web application 722 may include chat module 721A, and the doctor focused medical application 715 may include chat module 720A, each of which may expose windows, buttons, and other user engagement features of the respective chat module 720A, 721A to the respective web UI 752A or application UI 754A.

In an example, a patient at patient location 714 may include one or more computing devices 706, which may include a desktop computer, a laptop computer, a mobile phone, a tablet computer, and so on. In some embodiments, the computing device 706 uses a locally running web browser to access a web user interface (UI) 752B of patient-focused web application 724. Alternatively, or additionally, the computing device 706 may include patient-focused medical application 716 installed thereon, and may access the patient-focused medical application 716 via an application UI 754B of the patient-focused medical application 716. The patient-focused web application 724 may include chat module 721B, and the patient-focused medical application 716 may include chat module 720B, each of which may expose windows, buttons, and other user engagement features of the respective chat module 720B, 721B to the respective web UI 752B or application UI 754B.

In an example, a sales representative at sales representative location 716 may include one or more computing devices 707, which may include a desktop computer, a laptop computer, a mobile phone, a tablet computer, and so on. In some embodiments, the computing device 707 uses a locally running web browser to access a web user interface (UI) 752C of web application 726 (e.g., which may be a representative-focused application). Alternatively, or additionally, the computing device 707 may include a local application (Not shown) installed thereon that is designed for sales representatives, and may access the application via an application UI of the application. The web application 726 may include chat module 721C, which may expose windows, buttons, and other user engagement features of the chat module 721C to the respective web UI 752C.

In addition to presentation layer 772, the architecture 748 for the messaging platform includes a business logic layer 774. The business logic layer 772 is handles core functionality of the messaging platform. The business logic layer 772 includes all rules and logic of the messaging platform, and ensures that data is transmitted between different end devices (e.g., between computing devices 705, 706 and/or 707) and for storing data in a data layer (not shown).

In embodiments, the business logic layer includes an API gateway 756 and a plurality of APIs 759. API gateway 756 is a server or service that acts as an intermediary between clients (e.g., such as web applications 722, 724, 746 and/or mobile applications 715, 716) and a collection of backend services, each of which may include its own API. The API gateway 756 may be responsible for routing requests, aggregating responses, and managing various aspects of communications between clients and services. In some embodiments, the API gateway 756 validates the authentication of users of the messaging platform, and determines which users have access to communicate with which other users. One example of an API gateway 756 is a WSO2 Gateway.

In some embodiments, an API query language 758 is used to interface API gateway 756 with the APIs 759. The API query language 758 may be responsible for determining which users to connect with which other users based on received information, and for determining which APIs to invoke. The API query language 758 may enable clients to specify the structure of data when interacting with an API. In embodiments, the API query language 758 allows clients (e.g., web applications 722, 724, 726 and/or applications 715, 716) to declare the shape and structure of data to be sent to the clients, rather than relying on redefined responses, such as with traditional REST APIs. In one embodiment, the API query language 758 is Graph QL.

In embodiments, the APIs 759 include a chat API 760 and an encryption API 762. The APIs 759 may additionally include one or more other APIs, such as a doctors API, a user API, an assets API, an events API, and/or a notification API. More, fewer and/or different APIs may also be used.

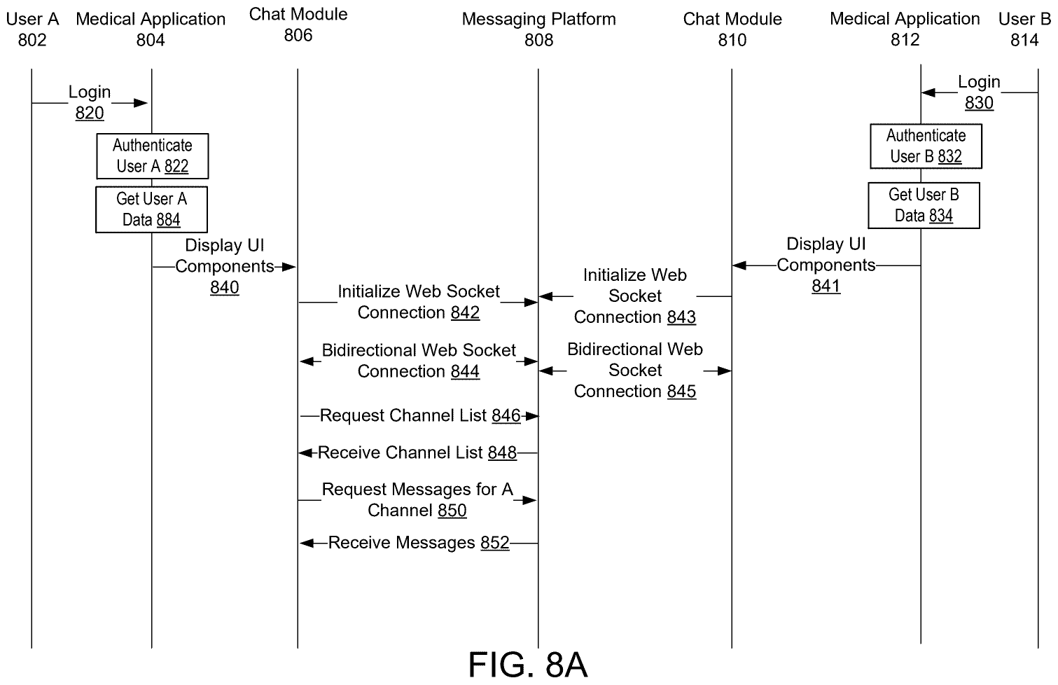

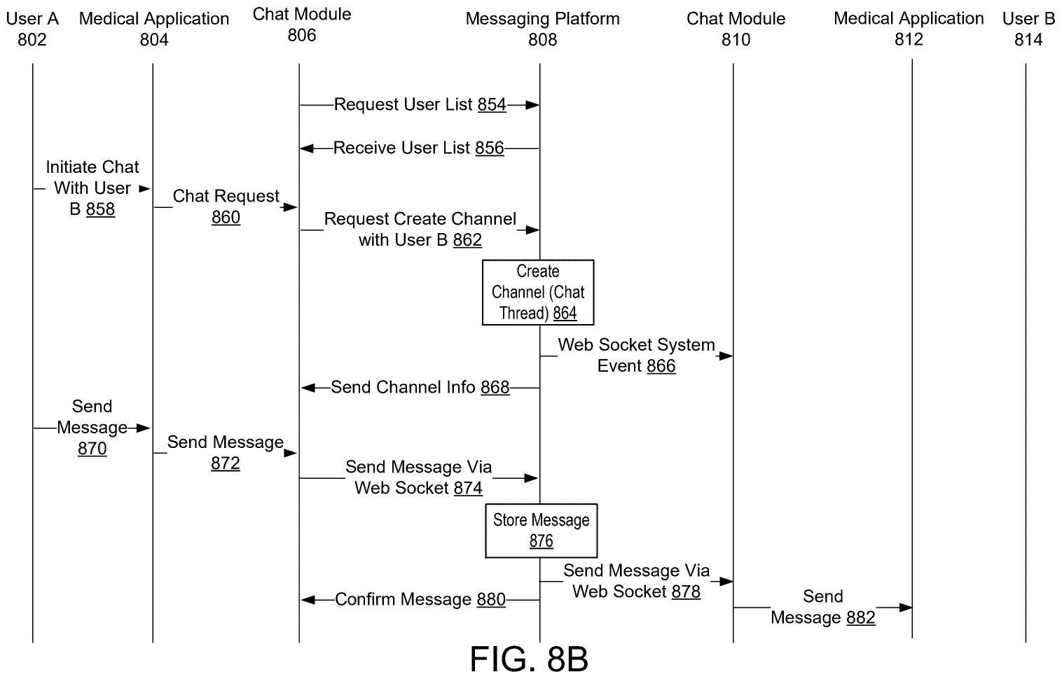

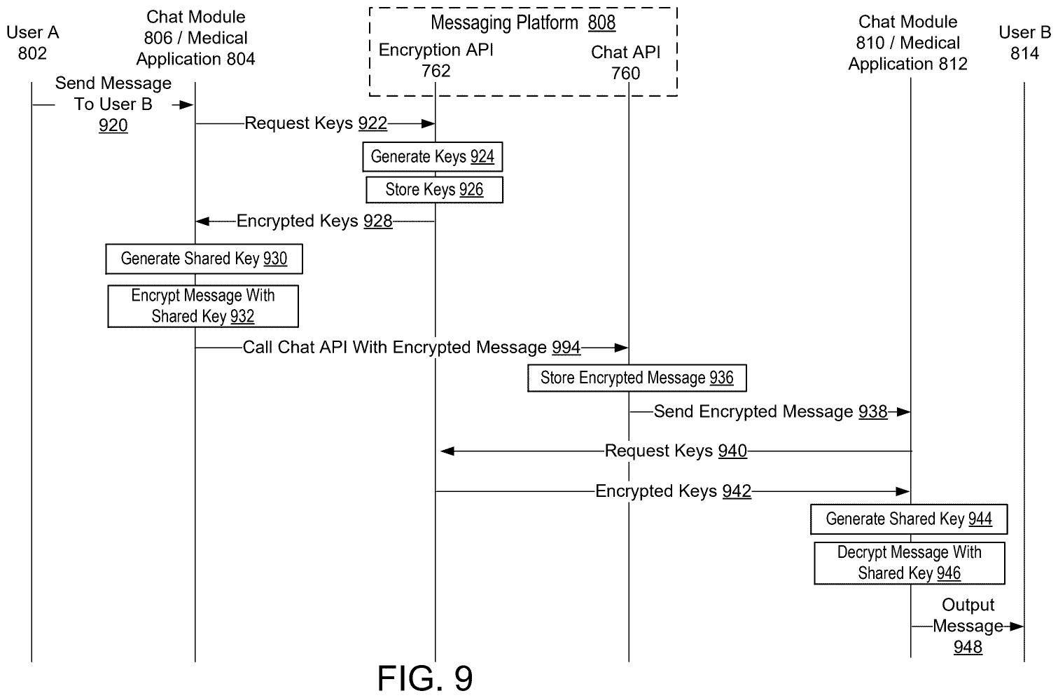

The chat API 760 may be responsible for establishing chat sessions between end users, handling the flow of messages between end users, saving messages, and so on. FIGS. 8A-B show certain operations of the chat API 760. The encryption API 762 handles encryption and decryption of messages between end users. FIG. 9 shows certain operations of the encryption API 762.

When users log into their respective instances of an application (e.g., a web application 722, 724, 726 or a local application 715, 716), the application to which the user has logged in may establish a connection with the business logic layer 774, which may run on one or more server computing devices. In embodiments, a websocket may be opened between the computing device 705, 706, 707 of the end user and the server computing device hosting the business logic layer 774. Once websockets are opened to the computing devices of two end users who are associated with a chat thread or message history, or for which one of the end users requests to establish a chat session with the other end user, a live chat session may be established between the computing devices of the two end users using the respective websocket connections associated with those computing devices. In embodiments, a data streaming layer 776 may include a managed event streaming component 780 that may be responsible for handling message streaming (e.g., between end users). The managed event streaming component 780 may enable the messaging platform (e.g., chat API 760 of the messaging platform) to stream large volumes of events or messages in real time. In embodiments, the managed event streaming component acts as a high-performance distributed message queue in which producers (e.g., message senders) publish messages to topics (e.g., message or chat threads set up between doctors and patients) and consumers subscribe to those topics to consume messages asynchronously. In one embodiment, the managed event streaming component 780 is implemented using Heroku Kafka.

FIG. 8A is a sequence diagram illustrating bidirectional communication between disparate medical applications via an application-agnostic messaging platform, in accordance with an embodiment. The sequence diagram shows a user A 802 (e.g., a doctor), a medical application 804, a chat module 806 associated with the medical application 804, a messaging platform 808, and a user B 814, a medical application 812 that is different from medical application 804, and a chat module 810 that is associated with medical application 812. In one embodiment, medical application 804 may correspond to one of doctor-focused medical application 715 or doctor-focused web application 722 and medical application 812 may correspond to one of patient-focused medical application 716 or patient-focused web application 724. Alternatively, medical application 804 may correspond to one of patient-focused medical application 716 or patient-focused web application 724 and medical application 812 may correspond to one of doctor-focused medical application 715 or doctor-focused web application 722. Alternatively, either of medical application 804 or 812 may be substituted by a non-medical application, such as application 717 or web application 726 of FIG. 7A. Messaging platform 808 may correspond to messaging platform 722 of FIG. 7A.

At block 820 of the sequence diagram, user A 802 logs in to medical application 804. This may include loading medical application 804 and providing credentials (e.g., username, password, personal identification number (PIN) and/or biometric data) to log into an account of user A on medical application 804. If user A provides proper authentication information, then medical application 804 may authenticate user A at block 822. Medical application 804 may then retrieve user A's data from local storage and/or from a remote data store (e.g., by querying a database) at block 824. The type of data available to user A may depend on medical application 804 and/or a role of user A in embodiments. For example, if user A is a doctor, then the user A data may include a list of patients of user A, medical information on user A's patients, a list of prospective-patients of user A, a list of message threads/histories between user A and other users (e.g., patients and/or sales representatives), and so on. If user A is a patient, then user A data may include medical information of user A, one or more doctors of user A, message threads to the one or more doctors of user A, and so on.

In some embodiments, the messaging platform 722 provides search functionality. For example, a user (e.g., doctor, doctor's staff, patient, etc.) may input a search for a particular patient name, a message date, etc., and messaging platform may perform a search on the requested information stored in a data store, such as a database. In some embodiments, searches are performed within the context of the requestor, and search results that the requestor is not associated with or not permitted to view are not returned.

Similarly, at block 830 user B may login to medical application 812. If user B provides proper authentication information, then medical application 812 may authenticate user B at block 832. Medical application 812 may then retrieve user B's data from local storage and/or from a remote data store (e.g., by querying a database) at block 834. In embodiments, when a user is authenticated to a respective medical application 804, 812, that user is also authenticated to messaging platform 808. The messaging platform 808 may rely on authentication performed at the medical application 804, 812, and may not require further authentication in order to access messaging platform 808.

From the medical application 804, user A 802 may select one or more UI elements that are associated with chat functionality provided by chat module 806. For example, the medical application may include a live chat button or messages button that, when selected, launches functionality of chat module 806.

In one embodiment, responsive to user A 802 selecting a UI element associated with chat module, a web socket connection 842 is initialized with messaging platform 808 (e.g., with server computing devices hosting messaging platform 808) by chat module 806. Alternatively, in some embodiments the web socket connection 842 may be initialized responsive to user A successfully logging into medical application 804. Once the web socket connection is initialized, at block 844A a bidirectional web socket connection 844 may be established between the chat module 806 and the messaging platform 808. The bidirectional web socket connection 844 may be a persistent connection that provides full-duplex communication (e.g., where the chat module and messaging platform can send and receive messages independently at any time).

Responsive to user A 802 selecting a UI element associated with chat module 806, one or more additional UI components associated with various functions of chat module 806 may be displayed at block 840. Such UI components may depend on a role of user A in embodiments and/or on medical application 804 in embodiments. If user A is a doctor, then UI components may include a tab, window or option for listing patients, and/or a tab, window or option for listing prospective patients.

In one embodiment, once the bidirectional web socket connection 844 is established between chat module 806 and messaging platform 808, at block 846 chat module requests a channel list 846 from messaging platform 808. Each channel may correspond to a message thread or history between user A and a different user (e.g., user B). Each channel may be for a distinct pair of users. Messaging platform 808 may store the channel list and all message histories in a data store (e.g., a database). Messaging platform 808 may perform a lookup for channels associated with user A, and may return a channel list at block 848.

In one embodiment, if the doctor selects the “patients” tab, then a list of patients of the doctor may be displayed. The list of patients may be retrieved from messaging platform 808 or from another system or data store in embodiments. The list of patients may be requested and returned in a similar manner to the channel list in embodiments.

The doctor may then select one of their patients from the list of patients. Once a patient is selected, chat module 806 may query messaging platform 808 for any prior message thread/history (e.g., a list of messages between the doctor and the selected patient for a channel associated with the doctor and the patient) at block 850. The message thread/history may be retrieved from a data store by messaging platform 808. Such a message thread/history for a channel may then be transmitted from messaging platform 808 to medical application 804 at block 852, after which the message thread/history may be displayed. A chat window may be shown, in which user A may type a message to user B.

The doctor may select a UI element/component (e.g., a button) to send a new message to the selected patient whether or not a prior message has been exchanged with the patient. If the patient has logged into a patient-focused medical application (e.g., medical application 812) that has an established bidirectional web socket connection to the messaging platform 808, then the message may be sent immediately to the computing device of the patient (e.g., live). If the patient's computing device does not have an active web socket connection to the messaging platform, then the message may be stored and may be sent to the device of the patient the next time that device connects to messaging platform 808. Additionally, in some cases an off-channel notification may be sent to the patient (e.g., via email, a social media post, a text message to a phone number of the patient, etc.) notifying the patient that they have a message from their doctor and that they can access the message by loading their patient-focused medical application.

As with medical application 804, medical application 812 may display UI components of chat module 810 at block 841 responsive to user B 814 successfully logging into medical application 812 and/or selecting chat module 810 from medical application 812. Additionally, chat module 810 may initialize a web socket connection 843 with messaging platform 808, and a bidirectional web socket connection 845 may be established between chat module 810 and messaging platform 808.

In the instant example, once chat module 806 and chat module 810 each have a bidirectional web socket connection established with messaging platform 808, a channel between user A and user B may become a live chat session in which communications between user A and user B over messaging platform 808 can be made live (e.g., in real time such that messages may appear on the device of user A as they are typed and/or entered in a device of user B, and vice versa).

FIG. 8B is an additional sequence diagram illustrating bidirectional communication between disparate medical applications via an application-agnostic messaging platform, in accordance with an embodiment. In embodiments, the sequence diagram of FIG. 8B shows operations that may be performed after the operations from the sequence diagram of FIG. 8A have been executed. In embodiments, the sequence diagram of FIG. 8B assumes that both user A 802 and user B 814 have already successfully logged into their respective medical applications 804, 812 and have established respective bidirectional web socket connections with messaging platform 808.

At block 854, chat module 806 may request a user list (e.g., list of users that user A is permitted to communication with, such as a list of patients or sales representatives for a doctor or a list of doctors for a patient). The user list may be requested, for example, responsive to user A successfully authenticating with medical application 804 in an embodiment. At block 856, messaging platform 808 may perform a lookup in a data store for user A, and may return a user list determined based on the lookup. Once a user list (e.g., patient list) is received by chat module 806, chat module 806 may display the user list, and user A 802 may select a user from the user list to communicate with.

At block 858, user A sends a command to initiate a chat with user B (where user B was selected from the user list). The medical application may then send a chat request 860 to chat module 806. If no channel previously existed between user A and user B, then chat module sends a request to messaging platform 808 to create such a channel with user B at block 862. At block 864, messaging platform 808 creates a channel (e.g., a chat thread/message thread) between user A and user B. A web socket system event for the created channel may be sent by messaging platform to chat module 810 at block 866. Messaging platform 808 may additionally send information on the created channel between user A and user B to chat module 806. Once the channel has been created, chat module 806 and chat module 810 may exchange messages via that channel. If chat module 806 and chat module 810 both have active bidirectional web socket connections to messaging platform 808, then such messages can be exchanged in real time or near real time (e.g., live).

At block 870, user A enters a message into medical application 804 (e.g., via typing the message or dictating the message). At block 872, medical application 804 sends the message to chat module 806. Chat module 806 may then send the message to messaging platform 808 via an open web socket at block 874. At block 876, messaging platform may store the message 876 in a data store. In embodiments, messages (e.g., conversations) may be archived, and can later be retrieved. Assuming chat module 810 has an open web socket connection to messaging platform 808, messaging platform 808 sends the message to chat module 810 via the web socket connection. Otherwise, messaging platform will wait until a web socket connection is established with chat module 810, and will send the message once the web socket connection is established. Chat module 810 may then send the message to medical application 812 at block 882, which may display the message to user B 814. Additionally, messaging platform 808 may send a message confirmation back to chat module 806 at block 880 indicating that the message has been delivered to chat module 810.

FIG. 9 is a sequence diagram illustrating secured bidirectional communication between disparate medical applications via an application-agnostic messaging platform, in accordance with an embodiment. In some embodiments, the operations of the sequence diagram of FIG. 9 is performed in conjunction with the operations of the sequence diagrams of FIG. 8A and/or 8B. Each message that is exchanged between two parties (e.g., user A 802 and user B 814) may relate to medical conditions, and so should be encrypted. Accordingly, encryption API 762 may be responsible for handling one or more encryption and/or decryption functions, such as generation and distribution of public and/or private keys and/or generation and/or distribution of shared keys. In embodiments, unique encryption keys (e.g., shared keys) are created for each conversation between two parties (e.g., between a doctor and a patient). A unique channel may be generated for each unique combination of two parties, and each channel may have its own encryption keys. In some embodiments, new encryption keys are created for each active channel or conversation on a periodic basis, such as every hour. This may include generating new public and private keys for the two parties of the channel and/or generating a new shared key for the channel.

In some embodiments, the sequence diagram of FIG. 9 shows operations that may be performed after the operations from the sequence diagram of FIG. 8A have been executed. In some embodiments, the sequence diagram of FIG. 9 assumes that both user A 802 and user B 814 have already successfully logged into their respective medical applications 804, 812 and have established respective bidirectional web socket connections with messaging platform 808. In FIG. 9, the chat module 806 and medical application 804 are combined into a single entity for simplification. Additionally, chat module 810 and medical application 812 have also been combined into a single entity for simplification.

At block 920, user A generates a message for sending to user B, where the message is provided to the chat module 806. At block 922, the chat module 806 requests keys from encryption API 762 of messaging platform 808. At block 924, encryption API 762 may generate public and private keys for both user A and user B. Alternatively, encryption API 762 may retrieve one or more of the keys from storage if they have already been created. At block 926, the encryption API 762 stores the keys in a secure data store. In embodiments, the keys are encrypted and stored in an encrypted state in the data store. At block 928, encryption API 762 sends one or more of the keys to the chat module 806 in an encrypted state. In one embodiment, the encrypted public and private keys of user A are sent to chat module 806, and the encrypted public key of user B is sent to chat module 806. Once the encrypted keys are received, chat module 806 may decrypt user A's private key and user B's public key. At block 932, chat module 806 may then generate a shared key for a channel between user A and user B using user A's private key and user B's public key. In some embodiments, encryption API 762 generates the shared key and sends the shared key to chat module 806 in an encrypted state, which may be decrypted by chat module 806. Note that the operations of blocks 922, 924, 926 and 928 may be omitted if chat module 806 already has local copies of user A's public and private keys and user B's public key. Additionally, the operations of block 930 may be omitted if a shared key for the channel between user A and user B has already been generated.

Once the shared key for the channel between user A and user B has been generated, at block 932 chat module 806 may encrypt the message to user B using the shared key. At block 934, the chat module 806 may call chat API 760 with the encrypted message, which may include sending the encrypted message to chat API 760. Chat API 760 stores the encrypted message at block 936. Chat API 760 additionally sends the encrypted message to chat module 810 via an open web socket connection.

At block 940, chat module 810 may request keys from encryption API 762 via a call to encryption API 762. At block 942, encryption API 762 may provide the requested keys to chat module 810 in encrypted form. In embodiments, chat module 810 receives user B's public and private keys and user A's public key in encrypted form. At block 944, chat module 810 may decrypt the encrypted keys and then generate at its end the shared key for the channel between user A and user B using user B's private key and user A's public key. In some embodiments, encryption API 762 generates the shared key and sends the shared key to chat module 810 in an encrypted state, which may be decrypted by chat module 810. Note that the operations of blocks 940 and 942 may be omitted if chat module 810 already has local copies of user B's public and private keys and user A's public key. Additionally, the operations of block 944 may be omitted if the shared key for the channel between user A and user B has already been generated at chat module 810.

At block 946, chat module 810 decrypts the encrypted message using the shared key. At block 948, the medical application 812 outputs the message in plain text form to user B 814. A similar process may also be performed for messages originating from user B and sent to user A. once both chat module 810 and chat module 806 have a copy of the shared key for the channel between user A and user B, messages may be encrypted, sent, and decrypted without performing any of the key retrieval and/or key generation operations.

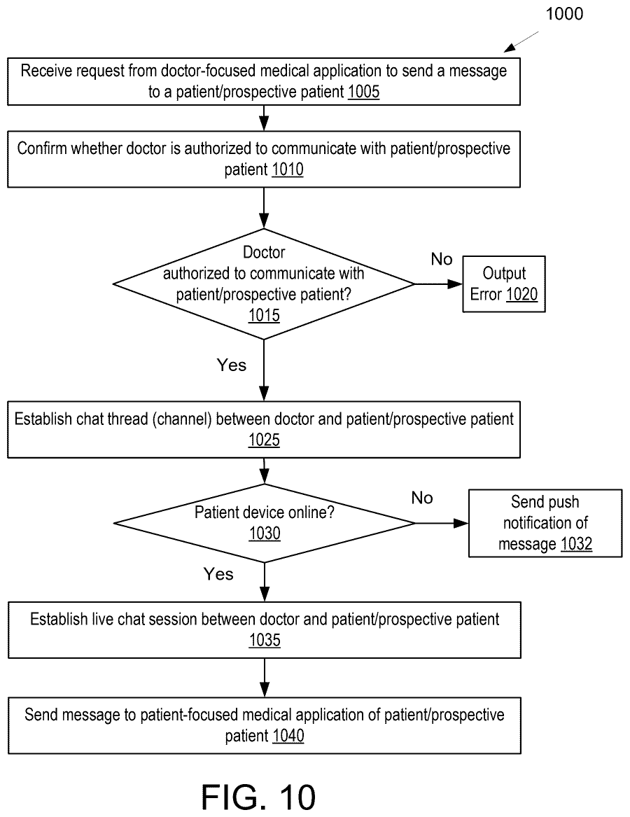

FIG. 10 is a flow diagram for a method 1000 of providing a live chat session between disparate medical applications, in accordance with an embodiment. Method 1000 may be performed by a processing logic that may comprise hardware (e.g., circuitry, dedicated logic, programmable logic, microcode, etc.), software (e.g., instructions run on a processing device to perform hardware simulation), firmware, or a combination thereof. In one embodiment, at least some operations of method 1000 are performed by one or more server computing device hosting a messaging platform (e.g., by server computing device(s) 708 of FIG. 7A).

In embodiments, method 1000 may be performed by a messaging platform to enable instant messaging between different parties using different types of applications (e.g., different types of medical applications), where each of those applications may include a chat module that interfaces with the messaging platform. At block 1005 of method 1000, processing logic receives a request from a doctor-focused medical application to send a message to a patient or prospective patient. At block 1010, processing logic may confirm whether the doctor is authorized to communicate with the patient or prospective patient. For example, doctors may be authorized to communicate with their own patients or patients of their practice, but may not be authorized to communicate with patients of other doctors. Processing logic may perform a lookup in a data store to determine whether the patient or prospective patient in question is associated with the doctor. If so, the doctor is authorized to communicate with the patient or prospective patient. At block 1015, if the doctor is authorized to communicate with the patient, the method proceeds to block 1025. Otherwise, the method proceeds to block 1020, and an error may be output.

At block 1030, processing logic determines whether a device of the selected patient (or prospective patient) is online (e.g., whether the messaging platform has an open web socket with the device of the selected patient). If the selected patient's device is online, the method continues to block 1035. Otherwise, the method continues to block 1032, and processing logic may send a push notification to one or more devices (and/or email addresses) of the patient indicating that they have a message from their doctor accessible via a patient-focused medical application. The push notification may include a text message, an email message, and/or other type of message in embodiments.

At block 1035, processing logic establishes a live chat session between the doctor and the patient or prospective patient using bidirectional web socket connections between the messaging platform and the device of the doctor and between the messaging platform and the device of the patient. At block 1040, processing logic sends the message to the patient-focused medical application of the patient or prospective patient. The patient or prospective patient may then view the message on the patient-focused medical application.

As described earlier, one or more functions of a medical application installed on a mobile device of a user (e.g., of a patient or doctor) may be voice activated. These functions may be voice activated whether or not the medical application is running on the mobile device in embodiments. Embodiments are described with reference to voice activated functions on a patient-focused medical application. However, it should be understood that the same techniques may be used to enable voice activated functions of a doctor-focused medical application.

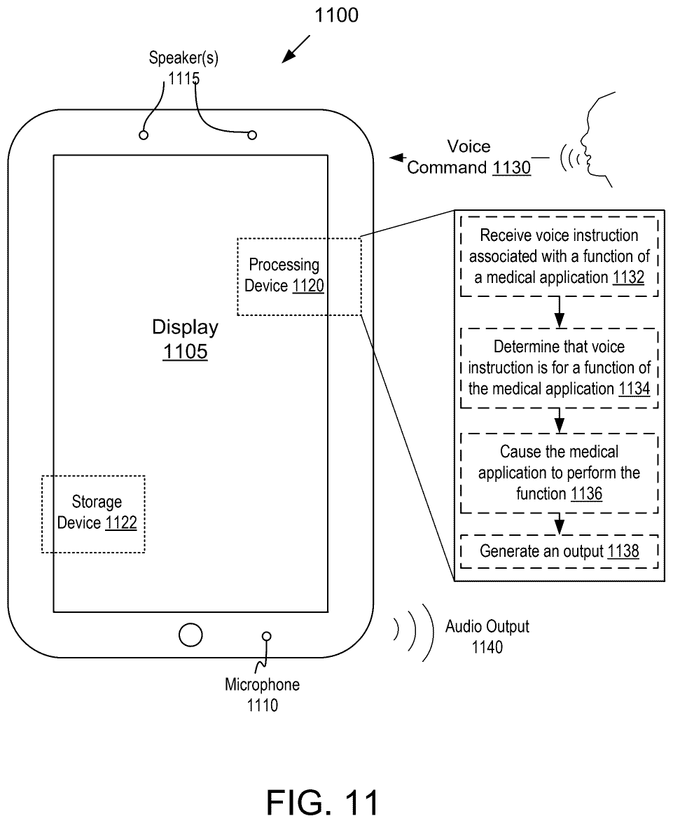

FIG. 11 illustrates a mobile device 1100 with a voice-controlled medical application installed thereon, in accordance with an embodiment. In embodiments, a user is able to operate and interface with a medical application in a hands free manner, increasing a convenience of using the medical application. The added voice controls may make the medical application more engaging and intuitive to use. Mobile device 1100 may correspond, for example, to computing device 705 or computing device 706 of FIG. 7A in embodiments. As shown, a mobile device 1100 may include one or more processing devices 1120, one or more storage devices 1122, one or more microphone 1110, one or more display 1105, and one or more speakers 1115, in addition to other components. A medical application (e.g., a patient-focused medical application) may be installed on the mobile device 1100, and one or more functions of the medical application may be registered with an application intents framework. Accordingly, a user may provide a command (e.g., a voice command or a button push) activating a listening mode of the mobile device 1100. Once the mobile device 1100 is in a listening mode, it may receive a voice command 1130 spoken by the user (e.g., a patient).

At block 1132, the processing device(s) 1120 may receive a voice instruction included in the voice command, where the voice instruction is associated with a function a the medical application installed on mobile device 1100. At block 1134, the processing device(s) 1120 determine that the voice instruction is for a function of the medical application. This may be determined based on one or more phrases and/or terms that are registered with the intents framework for the function of the medical application. For example, the phrase “start aligner timer” may be registered with the intents framework for a function of starting a timer that keeps track of an amount of time that a patient's orthodontic aligner has been removed from the patient's mouth. Many different functions may be voice activated, and each may be associated with its own phrase, word and/or set of phrases and/or words. An example of functions that may be voice activated include starting, stopping and/or setting a wear timer that counts how much time an aligner is removed from a patients mouth. An example of a function that may be voice activated includes unlocking a medical application (e.g., by providing a PIN or password via voice). An example of a function that may be voice activated includes providing an identification of a current aligner number. An example of a function that may be voice activated includes providing an indication of a next aligner change date. An example of a function that may be voice activated includes enabling search of different functionalities of the medical application that are available for voice command. For example, a user may request a list of the functions that may be voice controlled and the phrases to control those functions. Responsive to a voice command for such a list, the list may be shown in a display or output verbally. In one embodiment, a user may provide an audio input to search for a function (e.g., such as timer for patient-focused medical application), and a search result can be shown in a display of the mobile device. A user may select the result (e.g., by tapping on it), which may load the medical application to a screen/window associated with the requested function of the medical application.

In one example, a patient that wants to open a patient-focused medical application may provide a voice command stating, “can you open the orthodontic treatment application?” The mobile device may respond with, “please enter PIN to open the application.” The user may respond verbally with their PIN. The mobile device may enter the PIN without manual input from the user, and may unlock the medical application.

In another example, a patient may state, “start timer for aligner”. The mobile application may respond with, “select how long the timer should be set.” The user may respond with a verbal statement of the selected time, e.g., such as by stating “set for 30 minutes.” The mobile device may then set the timer for the selected time (e.g., 30 minutes), and may start counting down.

Other examples of functions of the medical application that may be requested verbally include requesting an appointment with a doctor, creating an event in an appointment calendar, accepting an instruction sent by a doctor, changing a current aligner to a specified value, changing a total number of aligners to a specified value, extending a next change date (for changing from a current aligner to a next aligner) by a specified amount of time (e.g., such as 2 days), sending a message to a doctor, sending photos to a doctor, canceling a subscription (e.g., to a retainer), and so on.

Processing logic may process the received voice instruction and determine a similarity value between the voice instruction and one or more registered phrases/terms each associated with different functions of the medical application and/or other applications installed on mobile device 1100. Processing logic may identify a function of the medical application associated with a highest similarity value, and at block 1136 may cause the medical application to perform the function.

At block 1138, processing logic may then generate an output, which may be output as audio output 1140 in some embodiments. The generated output may include a verbal and/or audio output (e.g., a statement that the requested function was performed) and/or an output to display 1105 (e.g., showing an active timer on display 1105).

In some embodiments, one or more functions of the medical application may be associated with widgets for the medical application. In such cases, the medical application may not be opened in order to perform the function. In such instances, the widget may perform the function on behalf of the medical application.

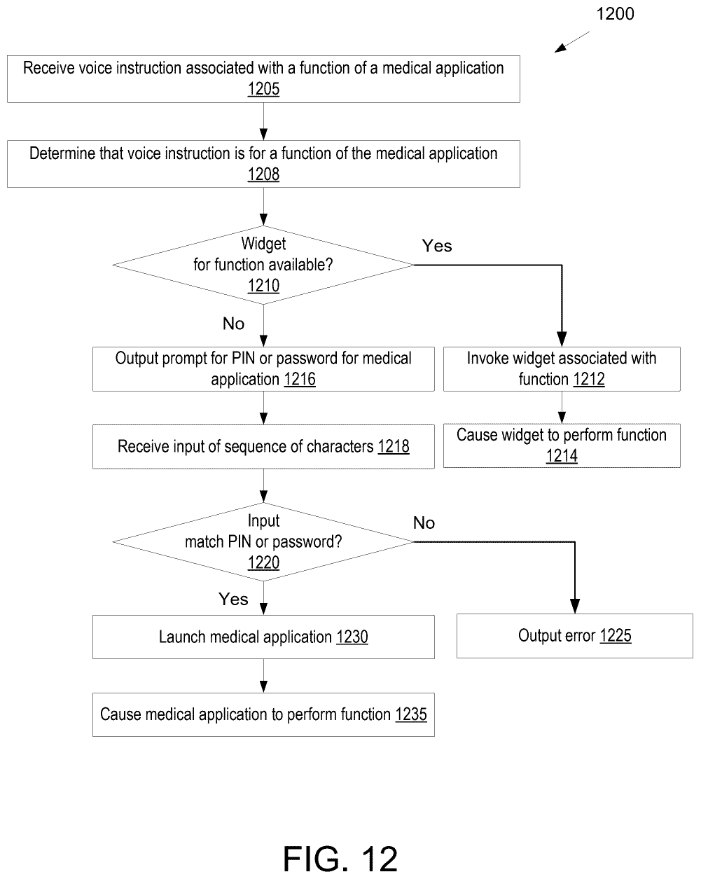

FIG. 12 is a flow diagram for a further method 1200 of providing voice control of a medical application, in accordance with an embodiment. Method 1200 may be performed by a processing logic that may comprise hardware (e.g., circuitry, dedicated logic, programmable logic, microcode, etc.), software (e.g., instructions run on a processing device to perform hardware simulation), firmware, or a combination thereof. Method 1200 may be performed, for example, by mobile device 1100 of FIG. 11 in some embodiments.

At block 1205 of method 1200, processing logic receives a voice instruction associated with a function of a medical application. At block 1208, processing logic determines that the voice instruction is for a function of the medical application.

At block 1210, processing logic determines whether a widget is available for the function of the medical application. If a widget is available, the method continues to block 1212. If not widget is available, the method proceeds to block 1216.

At block 1212, processing logic invokes the widget associated with the function. At block 1214, processing logic causes the widget to perform the function.

At block 1216, processing logic may output a prompt for a PIN or password (or optionally biometric information). The prompt may be output via a display and/or via audio. For example, an audio message asking the patient to speak their PIN or password may be output in embodiments. The medical application may include sensitive medical information of the patient, and so may require authentication of the patient in order to open. At block 1218, processing logic receives an input of a sequence of characters (or an input of biometric information, such as by a user looking into a camera or pressing a finger on a fingerprint scanner). In one embodiment, At block 1220, processing logic determines whether the input matches a PIN or password for the medical application (or matches stored biometric information of the user). If no match is found, the method may proceed to block 1225 and an error may be output. If a match is found, then at block 1230 processing logic may launch (e.g., load) the medical application. At block 1235, processing logic may then cause the medical application to perform the function.

FIG. 13 illustrates a diagrammatic representation of a machine in the example form of a computing device 1300 within which a set of instructions, for causing the machine to perform any one or more of the methodologies discussed herein, may be executed. In alternative embodiments, the machine may be connected (e.g., networked) to other machines in a Local Area Network (LAN), an intranet, an extranet, or the Internet. The computing device 1300 may correspond, for example, to computing devices 705-707 and/or server computing device(s) 709-711 of FIG. 7A. The computing device may alternatively or additionally correspond to local computing device 105 and/or remote server computing device 106 of FIG. 1. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein.

The example computing device 1300 includes a processing device 1302, a main memory 1304 (e.g., read-only memory (ROM), flash memory, dynamic random access memory (DRAM) such as synchronous DRAM (SDRAM), etc.), a static memory 1306 (e.g., flash memory, static random access memory (SRAM), etc.), and a secondary memory (e.g., a data storage device 1328), which communicate with each other via a bus 1308.

Processing device 1302 represents one or more general-purpose processors such as a microprocessor, central processing unit, or the like. More particularly, the processing device 1302 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 1302 may also be one or more special-purpose processing devices such as an application specific integrated circuit (ASIC), a field programmable gate array (FPGA), a digital signal processor (DSP), network processor, or the like. Processing device 1302 is configured to execute the processing logic (instructions 1326) for performing operations and steps discussed herein.

The computing device 1300 may further include a network interface device 1322 for communicating with a network 1364. The computing device 1300 also may include a video display unit 1310 (e.g., a liquid crystal display (LCD) or a cathode ray tube (CRT)), an alphanumeric input device 1312 (e.g., a keyboard), a cursor control device 1314 (e.g., a mouse), and a signal generation device 1320 (e.g., a speaker).

The data storage device 1328 may include a machine-readable storage medium (or more specifically a non-transitory computer-readable storage medium) 1324 on which is stored one or more sets of instructions 1326 embodying any one or more of the methodologies or functions described herein, such as instructions for chat model 1350, one or more ML models 1355, a search engine 1360, a chat module 1356, and/or a medical application 1351, which may correspond to similarly named components described above. A non-transitory storage medium refers to a storage medium other than a carrier wave. The instructions 1326 may also reside, completely or at least partially, within the main memory 1304 and/or within the processing device 1302 during execution thereof by the computer device 1300, the main memory 1304 and the processing device 1302 also constituting computer-readable storage media.

The computer readable storage medium 1324 may also store a software library containing methods for the chat model 1350, one or more ML models 1355, search engine 1360, chat module 1356, and/or medical application 1351. While the computer-readable storage medium 1324 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium other than a carrier wave that is capable of storing or encoding a set of instructions for execution by the machine and that cause the machine to perform any one or more of the methodologies of the present disclosure. The term “computer-readable storage medium” shall accordingly be taken to include, but not be limited to, solid-state memories, and optical and magnetic media.

Multiple example implementations are now provided.

Example 1: A method is described that includes receiving, by a user device, a prompt associated with treatment of a patient. It involves determining a treatment context for the treatment of the patient based on access to a back-end treatment context support system. The method further includes processing the prompt in view of the treatment context using one or more trained machine learning models to generate one or more actionable recommendations, and outputting the one or more actionable recommendations via at least one of the user device or an additional device associated with the user device.

Example 2: In the method of example 1, the prompt is received as an audio prompt via a microphone of the user device. The method further includes converting the audio prompt into a text prompt, wherein the text prompt is processed by the one or more trained machine learning models.

Example 3: In the method of example 2, the one or more actionable recommendations are generated in a text format. The method further includes performing text-to-speech conversion to convert the text format of the one or more actionable recommendations into a speech format, wherein the one or more actionable recommendations are output via a speaker of the user device.

Example 4: In the method of examples 1-3, determining the treatment context includes receiving one or more clues for the treatment context via at least one of the user device or an associated device, performing a lookup in a data store of the back-end treatment context support system for information on the treatment context based on the one or more clues, and retrieving the information from the data store.

Example 5: In the method of example 4, the data store comprises patient information for the patient.

Example 6: In the method of examples 4-5, the method further includes transmitting the prompt and the one or more clues from the user device to a server computing device associated with the back-end treatment context support system. The server computing device determines the treatment context and processes the prompt in view of the treatment context using the one or more trained machine learning models. The method also includes transmitting the one or more actionable recommendations from the server computing device to at least one of the user device or the additional device.

Example 7: In the method of examples 4-6, receiving the one or more clues for the treatment context includes at least one of capturing one or more images indicating a presence or lack of presence of the patient by the user device or the associated device, or capturing audio indicating the presence or lack of presence of the patient. The content of the one or more actionable recommendations is based at least in part on the presence or lack of presence of the patient.

Example 8: In the method of examples 4-7, the user device comprises an intraoral scanner, and receiving the one or more clues for the treatment context includes receiving at least one of image data captured by the intraoral scanner or movement data of the intraoral scanner.

Example 9: In the method of examples 4-8, the method further includes encrypting communications between the user device and a server computing device that performs the determining of the treatment context and the processing of the prompt.

Example 10: In the method of example 1-9, the actionable recommendations comprise at least one of treatment information, digital practice management information, or doctor-patient relationship information.

Example 11: In the method of examples 1-10, the one or more actionable recommendations are in a format not supported by the user device and are output to the additional device. The method includes outputting a signal via the user device indicating that a message comprising the one or more actionable recommendations is available.

Example 12: In the method of examples 1-11, the treatment context comprises at least one of a clinical context, a patient-doctor interaction context, or a medical practice management context.

Example 13: In the method of examples 1-12, determining the treatment context includes determining an instance of a medical application in use for the patient. The one or more trained machine learning models further output one or more control instructions for controlling the instance of the medical application. The method further includes controlling the instance of the medical application using the one or more control instructions, wherein the controlling is performed in response to the prompt.

Example 14: In the method of example 13, the prompt is with regards to a treatment plan for at least one of orthodontic treatment or restorative dental treatment presented by the instance of the medical application. The actionable recommendations comprise at least one of information about an aspect of the treatment plan, an explanation of reasons for the aspect of the treatment plan, or instructions on how to use the medical application.

Example 15: In the method of examples 1-14, the user device comprises a wearable internet-of-things (IoT) device, an intraoral scanner, a mobile computing device, a wearable augmented reality (AR) device, or a smart speaker.

Example 16: In the method of examples 1-15, the prompt comprises a request for images of prior patients having particular characteristics. The one or more actionable recommendations comprise identifiers for one or more pre-treatment images of prior patients having the particular characteristics. The method further includes retrieving the one or more pre-treatment images by the back-end treatment context system, determining one or more post-treatment images associated with the one or more pre-treatment images, and outputting the one or more pre-treatment images and the one or more post-treatment images to the additional device for display to the patient.

Example 17: In the method of examples 1-16, the treatment context comprises at least one of a medical application or a medical product. The method further includes determining that the one or more actionable recommendations are insufficient to address one or more questions in the prompt, and establishing a voice connection between the user device and a representative of the medical application or the medical product.

Example 18: In the method of examples 17, the method further includes selecting the representative from a plurality of representatives based on a determination that the representative will be able to answer the one or more questions.

Example 19: In the method of examples 1-18, the one or more trained machine learning models comprise a large language model (LLM) trained on historical medical treatment histories of a plurality of prior patients.

Example 20: In the method of examples 1-19, the method further includes generating, by the one or more trained machine learning models, one or more search queries to a search engine based on the prompt, providing the one or more search queries to the search engine by the back-end treatment context support system, and receiving the treatment context from the search engine responsive to providing the one or more search queries to the search engine.

Example 21: In the method of examples 1-20, the method further includes receiving information pertaining to the treatment context from at least one of an intraoral scanning application, a treatment planning application, or a treatment monitoring application, wherein the received information is used to determine the treatment context.

Example 22: A system comprising a memory and a computing device is configured to perform the method of any of examples 1 through 21.

Example 23: A computer readable medium comprises instructions that, when executed by a processing device, cause the processing device to perform the method of any of examples 1 through 21.

Example 24: A system is described that includes a local computing device configured to execute a medical application for a patient, and a user device comprising a microphone and a speaker. The user device is configured to capture a prompt associated with treatment of the patient. A server computing device is configured to receive the prompt from the user device, receive one or more clues about a treatment context from at least one of the user device or the local computing device, determine the treatment context for the patient based on the one or more clues, process the prompt in view of the treatment context using one or more trained machine learning models to generate one or more actionable recommendations, and send the one or more actionable recommendations to at least one of the user device or the local computing device, wherein the one or more actionable recommendations are output via at least one of the user device or the local computing device.

Example 25: A system includes a server computing device with a messaging platform that facilitates communication between various types of medical applications. It also includes a first computing device used by a doctor, which has a doctor-focused medical application and a first chat module integrated with the doctor-focused medical application, enabling the doctor-focused medical application to interface with the messaging platform. Additionally, it includes a second computing device used by a patient or prospective patient, which has a patient-focused medical application and a second chat module that integrates with the patient-focused medical application, allowing the patient-focused medical application to interface with the messaging platform. The patient-focused medical application has different functionality than the doctor-focused medical application. The messaging platform is configured to send messages between the doctor-focused and patient-focused medical applications.

Example 26: The system of example 25 where the server computing device is configured to receive a request from the doctor-focused medical application to send a message to the patient or prospective patient, conform whether the doctor is authorized to communicate with the patient or prospective patient and, upon confirmation, establish a chat thread between the doctor-focused and patient-focused medical applications.

Example 27: The system of example 26 where the server computing device is further configured to determine if the patient-focused medical application is connected to the server computing device. If it is, the server computing device opens a web socket to the second computing device and establishes a live chat session between the doctor's and the patient's computing devices (i.e., the first and second computing devices).

Example 28: The system of examples 26-27 where the server computing device is further configured to determine if the patient-focused medical application is not connected. If it is not, the server sends at least one of a push notification to the second computing device or an email notification to the email account of the patient or prospective patient, informing them that an unread message is available on the messaging platform.

Example 29: The system of example 28 where the server computing device is further configured to receive the push notification, output the push notification to a display of the second computing device, receive a command to launch the patient-focused medical application, and, after authenticating the patient or prospective patient, retrieve the message.

Example 30: The system of examples 26-29 where the server computing device is further configured to receive credentials from a doctor during a login attempt, authenticate the doctor based on those credentials, query the messaging platform for a list of patients and prospective patients the doctor is permitted to communicate with, and display one or both lists in the user interface of the doctor-focused medical application.

Example 31: The system of example 30 where the server computing device is further configured to receive a selection of a patient or prospective patient, determine whether a chat thread has already been established, display existing messages if the thread exists, or establish a new chat thread if it does not.

Example 32: The system of examples 25-31 where the server computing device is further configured to capture one or more facial images of the patient via the patient-focused medical application and attach them to a message to the doctor. The server computing device then transmits the message with the images to the first computing device for display in the doctor-focused medical application.

Example 33: The system of examples 25-32 where the server computing device is further configured to capture facial images of the patient via the patient-focused medical application, send them to the second computing device using a platform other than the messaging platform, and send a message via the messaging platform identifying the images.

Example 34: The system of examples 25-33 where the server computing device is further configured to present a gallery of facial images in the doctor-focused medical application, receive a selection of images, generate a message with the selected images and comments, and send the message to the messaging platform for delivery to the second computing device.

Example 35: The system of examples 25-34 in which both the first and second computing devices can encrypt outgoing messages and decrypt incoming messages using the messaging platform.

Example 36: The system of example 35 where the server computing device is further configured to generate public key pairs for the doctor and patient, create a shared key using these pairs, and send the shared key to both computing devices, with a unique key for each doctor-patient combination.

Example 37: The system of examples 25-36 where the server computing device is further configured to receive a search query from the first computing device, search message threads between the doctor and patients or prospective patients, and return the search results for display in the doctor-focused medical application.

Example 38: The system of examples 25-37 includes a third computing device used by a medical sales representative, which has an additional application and a third chat module that interfaces with the messaging platform.

Example 39: The system of examples 25-38 uses a hybrid cross-platform chat module for both the first and second chat modules.

Example 40: A mobile computing device of a patient includes a storage device with instructions for a medical application, a microphone, and one or more processing devices configured to receive a voice instruction associated with a function of the medical application via the microphone, determine that the voice instruction is for the function of the medical application, cause the medical application to perform the function, and generate an output responsive to causing the medical application to perform the function.

Example 41: The mobile computing device of example 40 uses a patient-focused orthodontic treatment application as the medical application.

Example 42: The mobile computing device of examples 40-41 identifies a widget associated with the function, invokes it if installed, and causes the medical application to perform the function.

Example 43: The mobile computing device of examples 40-42 outputs an audio prompt for a PIN or password, receives a verbal input, verifies it, and performs the function if the input matches the PIN or password.

Example 44: The mobile computing device of examples 40-43 includes a speaker, and the output is a verbal report that the function was performed.

Example 45: The mobile computing device of examples 40-44 includes a timer function for tracking how long an orthodontic aligner has been removed, and the voice instruction can set, start, or stop the timer.

Example 46: The mobile computing device of example 45 determines when the timer elapses and outputs an alarm via the speaker.

Example 47: The mobile computing device of examples 40-46 includes a function to launch the medical application via voice instruction.

Example 48: The mobile computing device of examples 40-47 includes an orthodontic aligner tracker that identifies the current aligner in use based on a voice request and outputs the identification.

Example 49: The mobile computing device of examples 40-48 includes an orthodontic aligner tracker that provides information on when to replace the current aligner based on a voice request.

Example 50: The mobile computing device of examples 40-49 includes a doctor finder function that responds to a voice request by displaying information about nearby orthodontic doctors.

Example 51: The mobile computing device of examples 40-50 includes a patient smile image capture function that launches upon a voice request and displays its user interface.

Example 52: The mobile computing device of example 51 captures an image of the patient's dentition and transmits it to a doctor's remote computing device.

Example 53: The mobile computing device of examples 51-52 captures a current smile image, compares it to a past image or sends it for comparison, and displays the resulting observations.

Example 54: The mobile computing device of examples 51-53 captures a current smile image, sends it for segmentation, receives segmentation information, and displays it.

Example 55: The mobile computing device of examples 40-54 is a mobile phone.

Example 56: The mobile computing device of examples 40-55 includes an appointment scheduling function that interfaces with a doctor's remote computing device upon a voice request.

Example 57: The mobile computing device of example 56 creates a calendar event for the scheduled appointment.

Example 58: The mobile computing device of examples 40-57 includes a messaging function that sends a dictated message to a doctor's remote computing device.

Example 59: The mobile computing device of examples 40-58 includes a speaker that outputs an audio message as the output.

It is to be understood that the above description is intended to be illustrative, and not restrictive. Many other embodiments will be apparent upon reading and understanding the above description. Although embodiments of the present disclosure have been described with reference to specific example embodiments, it will be recognized that the disclosure is not limited to the embodiments described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

1. A system comprising:
a user device comprising a memory and one or more processors, the user device configured to:
receive a prompt associated with treatment of a patient;
cause a treatment context for the treatment of the patient to be determined based on access to a back-end treatment context support system;
cause the prompt to be processed in view of the treatment context using one or more trained machine learning models to generate one or more actionable recommendations; and
output the one or more actionable recommendations via at least one of the user device or an additional device associated with the user device.

2. The system of claim 1, wherein the prompt is received as an audio prompt via a microphone of the user device, and wherein the user device is further configured to:
convert the audio prompt into a text prompt, wherein the text prompt is processed by the one or more trained machine learning models.

3. The system of claim 2, wherein the one or more actionable recommendations are generated in a text format, the system further comprising:
one or more server computing devices, configured to:
determine the treatment context and process the prompt in view of the treatment context;
perform text-to-speech conversion to convert the text format of the one or more actionable recommendations into a speech format; and
transit the speech format of the one or more actionable recommendations to the user device, wherein the one or more actionable recommendations are output via a speaker of the user device.

4. The system of claim 1, further comprising:
a server computing device associated with the back-end treatment context support system and configured to determine the treatment context, wherein determining the treatment context comprises:
receiving one or more clues for the treatment context via at least one of the user device or an associated device;
performing a lookup in a data store of the back-end treatment context support system for information on the treatment context based on the one or more clues; and
retrieving the information from the data store.

5. The system of claim 4, wherein the data store comprises patient information for the patient.

6. The system of claim 4, wherein:
the user device is configured to transmit the prompt and the one or more clues to the server computing device; and
the server computing device is configured to:
determine the treatment context and process the prompt in view of the treatment context using the one or more trained machine learning models; and
transmit the one or more actionable recommendations to at least one of the user device or the additional device.

7. The system of claim 4, wherein receiving the one or more clues for the treatment context comprises at least one of:
capturing one or more images indicating a presence or lack of presence of the patient by the user device or the associated device; or
capturing audio indicating the presence or lack of presence of the patient;
wherein content of the one or more actionable recommendations is based at least in part on the presence or lack of presence of the patient.

8. The system of claim 4, wherein the user device comprises an intraoral scanner, and wherein receiving the one or more clues for the treatment context comprises receiving at least one of image data captured by the intraoral scanner or movement data of the intraoral scanner.

9. The system of claim 4, wherein the user device is configured to:
encrypt communications between the user device and a server computing device that performs the determining of the treatment context and the processing of the prompt.

10. The system of claim 1, wherein the actionable recommendations comprise at least one of treatment information, digital practice management information, or doctor-patient relationship information.

11. The system of claim 1, wherein the one or more actionable recommendations are in a format not supported by the user device and are output to the additional device, and wherein a signal is to be output via the user device indicating that a message comprising the one or more actionable recommendations is available.

12. The system of claim 1, wherein the treatment context comprises at least one of a clinical context, a patient-doctor interaction context, or a medical practice management context.

13. The system of claim 1, wherein determining the treatment context comprises determining an instance of a medical application in use for the patient, and wherein the one or more trained machine learning models further output one or more control instructions for controlling the instance of the medical application, and wherein the instance of the medical application is to be controlled using the one or more control instructions, wherein the controlling is to be performed in response to the prompt.

14. The system of claim 13, wherein the prompt is with regards to a treatment plan for at least one of orthodontic treatment or restorative dental treatment presented by the instance of the medical application, and wherein the actionable recommendations comprise at least one of information about an aspect of the treatment plan, an explanation of reasons for the aspect of the treatment plan, or instructions on how to use the medical application.

15. The system of claim 1, wherein the user device comprises a wearable internet-of-things (IoT) device, an intraoral scanner, a mobile computing device, a wearable augmented reality (AR) device, or a smart speaker.

16. The system of claim 1, wherein the prompt comprises a request for images of prior patients having particular characteristics, and wherein the one or more actionable recommendations comprise identifiers for one or more pre-treatment images of prior patents having the particular characteristics, the system further comprising a server computing device configured to:
retrieve the one or more pre-treatment images by the back-end treatment context system;
determine one or more post-treatment images associated with the one or more pre-treatment images; and
output the one or more pre-treatment images and the one or more post-treatment images to the additional device for display to the patient.

17. The system of claim 1, wherein the treatment context comprises at least one of a medical application or a medical product, the system further comprising a server computing device configured to:
determine that the one or more actionable recommendations are insufficient to address one or more questions in the prompt; and
establish a voice connection between the user device and a representative of the medical application or the medical product.

18. The system of claim 1, wherein the one or more trained machine learning models comprise a large language model (LLM) trained on historical medical treatment histories of a plurality of prior patients.

19. The system of claim 1, further comprising a server computing device configured to:
generate, using the one or more trained machine learning models, one or more search queries to a search engine based on the prompt;
provide the one or more search queries to the search engine via the back-end treatment context support system; and
receive the treatment context from the search engine responsive to providing the one or more search queries to the search engine.

20. A system comprising:
a local computing device configured to execute a medical application for a patient;
a user device comprising a microphone and a speaker, the user device configured to:
capture a prompt asking associated with treatment of the patient; and
a server computing device configured to:
receive the prompt from the user device;
receive one or more clues about a treatment context from at least one of the user device or the local computing device;
determine the treatment context for the patient based on the one or more clues;
process the prompt in view of the treatment context using one or more trained machine learning models to generate one or more actionable recommendations; and
send the one or more actionable recommendations to at least one of the user device or the local computing device, wherein the one or more actionable recommendations are output via at least one of the user device or the local computing device.
